(19) United States
(12) Patent Application Publication
(10) Pub. No.: US 2026/0083683 A1
(43) Pub. Date: Mar. 26, 2026

(54) PROTEIN NANOSPHERES AND METHOD TO TREAT DYSFUNCTION FROM CHEMOTHERAPY AND IMMUNOSUPPRESSIVE THERAPY

(71) Applicant: Richard C.K. Yen, Yorba Linda, CA (US)

(72) Inventor: Richard C.K. Yen, Yorba Linda, CA (US)

(21) Appl. No.: 19/409,987

(22) Filed: Dec. 5, 2025

Related U.S. Application Data
(63) Continuation-in-part of application No. 19/190,246, filed on Apr. 25, 2025.
(63) Continuation-in-part of application No. 18/812,320, filed on Aug. 22, 2024.
(63) Continuation-in-part of application No. 18/735,523, filed on Jun. 6, 2024.
(63) Continuation-in-part of application No. 18/295,829, filed on Apr. 4, 2023.
(60) Provisional application No. 63/100,670, filed on Mar. 25, 2020.
(60) Provisional application No. 63/372,883, filed on Apr. 13, 2022.
(60) Provisional application No. 63/628,513, filed on Jul. 31, 2023.
(60) Provisional application No. 63/630,952, filed on Mar. 18, 2024.
(60) Provisional application No. 63/833,529, filed on Dec. 12, 2024.
(60) Provisional application No. 63/834,098, filed on Feb. 21, 2025.

(51) Int. Cl. A61K 9/51; A61K 9/00; A61K 38/36; A61K 47/20; A61P 37/04

(52) U.S. Cl. CPC A61K 9/5169; A61K 9/0019; A61K 38/363; A61K 47/20; A61P 37/04

(57) ABSTRACT

A protein nanospheres and method to treat dysfunction from chemotherapy and immunosuppressive therapy, and a manufacture of fibrinogen-coated albumin spheres (FAS) and High-Fibrinogen Spheres (HFS) which have higher concentrations of fibrinogen molecules per sphere than FAS, and their use for medical treatments. Both kinds of nanoparticles are effective in the mitigation of the toxic effects of certain chemotherapeutic and radiological agents that are typically used in the treatment of cancer, or the treatment of autoimmune diseases, or for patients with both diseases. FAS and HFS can exert their beneficial effects via a variety of mechanisms which match the need of the body for specific cell types, including any of the subgroups of T cells and antibody producing cells, the relative concentration of each kind is vital to the balance between tumor surveillance and autoimmune disease suppression.

### CROSS-REFERENCE TO RELATED APPLICATION

This application claims the benefit of priority under 35 U.S.C. § 119(e) based upon co-pending U.S. provisional patent application Ser. No. 63/833,529 filed on Dec. 9, 2024, and co-pending U.S. provisional patent application Ser. No. 63/834,098 filed on Feb. 18, 2025.

This application is a continuation-in-part under 35 U.S.C. § 120 based upon co-pending U.S. patent application Ser. No. 18/812,320 filed on Aug. 22, 2024, co-pending U.S. patent application Ser. No. 18/295,829 filed on Apr. 4, 2023, co-pending U.S. patent application Ser. No. 18/735,523 filed on Jun. 6, 2024, and co-pending U.S. patent application Ser. No. 19/190,246 filed on Apr. 25, 2025.

The entire disclosure of the prior provisional applications are incorporated herein by reference.

### BACKGROUND

### Technical Field

In some aspects, the present technology relates to a protein nanospheres and method to treat dysfunction from chemotherapy and immunosuppressive therapy for use in connection with mobilizing a patient's own stem cells and other mechanisms to mitigate harm caused by agents with multiple functions or targets. In some other aspects, the present technology relates to methods associated with mitigating harm caused by agents with multiple functions or targets, including those used in chemotherapy, immunosuppressive therapy and radiation therapy.

### Background Description

Chemotherapy is the treatment of cancer patients by using drugs (chemotherapeutic agents) to preferentially kill the cancer cells with the hope that healthy cells are mostly spared. Immunosuppressive therapy is the administration of drugs that suppress the immune system of a patient who has inappropriate immune responses which produce damage to the patient's own cells or tissues—however, the immunosuppressive therapy can by itself cause additional and unwanted harm to the patient.

Certain drugs can serve as both chemotherapeutic agents and immunosuppressive agents, thus they target more than one tissue or entity (such as cancer cells, overactive immune cells or infectious agents.) When two targets are identified, these drugs are often called dual-use agents. When more than two targets are known or suspected, they can be called multi-valent agents or “multi-agents.” Although typically used in different patient populations (e.g. one with cancer, the other with autoimmune or related immune disease) some such drugs are sometimes used in the same patient who has more than one health problem. One such drug is cyclophosphamide (CTX). CTX has many side effects including nausea, vomiting, low blood cells counts and myelosuppression. See: Gallicchio, “Recovery in vivo of murine hematopoietic stem cells after single-dose administration of cyclophosphamide” Exp Hematol 1986:14:395-400.

The side effects of CTX can be seen in over 30% of all treated patients. See: International Weidenstrom's Macroglobulnemia Foundation (IWNF) Cyclophosphamide Fact Sheet (updated Feb. 17, 2021). A summary of the common side effects of CTX and the mitigative methods for these CTX side-effects is presented below in Table 1.

TABLE 1
Various side effects of cyclophosphamide and current
treatment methods for such side effects

| | PERCENT | | |
|---|---|---|---|
| | OF | MITIGATION | |
| | PATIENTS | METHOD OR | |
| SIDE EFFECT | AFFECTED | TREATMENT | REFERENCE |
| Hemorrhagic | 25-60% | Hydration, mesna, | BJU Int 2023 |
| Cystitis | | bladder irrigation, | doi: 10.1111/bju.16140 Review |
| | | hyperbaric oxygen | Haemorrhagic cystitis: a review of |
| | | therapy | management strategies and |
| | | | emerging treatments Kevin D. Li |
| Myelosuppression | 20-25% | Growth factor (G- | Basic & Clinical Pharmacology & |
| | | CSF), dose | Toxicology, 2016, 119, 428-435 |
| | | adjustment, blood | Doi: 10.1111/bcpt.12600 |
| | | transfusion | Optimized Animal Model of |
| | | | Cyclophosphamide-induced Bone |
| | | | Marrow Suppression Lizhi Feng |
| Cardiotoxicity | 1-19% | Cardioprotective | Journals.plos.org Jun. 26, 2015. |
| | | agents (e.g. | Mechanisms of Fatal |
| | | dexrazoxane), | Cardiotoxicity following High- |
| | | antioxidants (e.g. N- | Dose Cyclophosphamide Therapy |
| | | acetylcysteine), | and a Method for Its Prevention. |
| | | regular monitoring | Takuro Nishikawa |
| Secondary | 2.8% | Regular monitoring, | Blood (2013) 122 (21): 5373. |
| Malignancies | | dose adjustment, | Cyclophosphamide Maintenance |
| | | alternative therapies | Therapy Is a Safe and Effective |
| | | | Alternative In Multiple Myeloma |
| | | | Patients Who Undergo Autologous |
| | | | Stem Cell Transplantation |
| | | | Michael T. Byrne |

It can be readily seen that all of the above “treatment” or “mitigative” methods do not address the fundamental issue of host cell destruction (these cells are needed for good health) by the drug. Stem cell therapy may be contemplated to mitigate the harmful effects of drugs such as CTX, but the search for a suitable donor (related or unrelated) is difficult and the timing of the administration of donor stem cells can be difficult to assess: if donor stem cells are administered too early relative to the optimal time frame, CTX may kill the infused stem cells as well as the endogenous cells; if donor stem cells are administered too late, the harmful effects of CTX may not be overcome by such exogenous stem cell therapy. Therefore, there is an urgent need for a solution that will offer “replacement of host cells needed for healthy life” or “regeneration of host's stem cells into cells for healthy life wherever they are needed in vivo.”

Recent discoveries in stem cell therapies have encouraged the use of stem cells obtained from the patient or from eligible donors. However, the results are often equivocal and the expenses are high. Also, there is a real risk of the injected stem cells developing into tumors in the host. Therefore, there is a need for new therapy involving the mobilization of the patient's own stem cells towards the mitigation of the harm caused by CTX or similar drugs, thus avoiding the known problems of using stem cells from sources other than the patient himself.

Yen has disclosed data showing that certain protein nanoparticles or nanospheres called Fibrinogen-coated Albumin Spheres (FAS) can be administered by way of the intravenous route, which will result in the mobilization of the stem cells toward the healing of wounded tissues. The process included the attachment of such nanoparticles to the cells residing in the endothelium of the blood vessels or to the bone marrow cells inside the bone marrow. Furthermore, administration of FAS intravenously has been shown to increase CD34+(stem) cells inside the bone marrow, followed by increased concentrations of certain “more mature” cells in the peripheral blood, such as monocytes and granulocytes. The prior disclosures include a U.S. non-provisional patent application titled “Protein Nanospheres to Treat Harm from Multiple Trauma.”

Specifically, a dose of Fibrinoplate-S(FPS, the name of the drug product, containing the active substance FAS) administered intravenously has been shown to result in the entry of FAS into the bone marrow compartment of the bone marrow. In some experiments, the FAS can be pre-labeled with a fluorescent compound called Fluorescein IsoThioCyanate (FITC). Following isolation of bone marrow cells from the bone marrow, FITC-labeled FAS can be seen to still attach to the isolated bone marrow cells. In separate experiments, unlabeled (original) FAS administered intravenously to animals have been shown to result in an increase of CD34+ cells in the bone marrow. Subsequently, an increased concentration of CD34+ cells can also be detected in the blood. As for a deep wound, as late as weeks after the administration of FPS, CD34+ cells can still be detected at the destination, resulting in the rapid healing of the wound. Such deep wounds include wounds caused by high dose of local irradiation to the skin (“irradiation-induced skin injury”) or bone fractures. However, there are no data in the public literature or patent applications that have shown that FAS can mobilize stem cells which can be beneficial for the treatment of tissues harmed by multi-valent drugs (also called multi-agents in this application). One example of such chemotherapy/immunotherapy drug is CTX which can be used as a single treatment entity toward the treatment of cancer or immunodysfunction; or as dual-use agent for patients who have both immunodysfunction and cancer (which is caused either by independent causes or as a result of immunotherapy.)

Given the lack of effectiveness of various prior arts to mitigate the damage caused by chemotherapy and/or immunotherapy drugs, a more effective (and easier-to-use) method of using stem cells from the patient to mitigate the harmful effects of mono-valent or multi-valent drugs is much needed. In this disclosure, we focus on the mechanism by the nanoparticles to mobilize stem cells: however, the nanoparticles may have more than one mechanism of action, which will be further discussed and included in this disclosure.

U.S. Pat. No. 11,246,877 B2 entitled “Nanoparticles for chemotherapy, targeted therapy” discloses nanoparticles combined with immunotherapy agents (not fibrinogen) for treating cancer-related conditions, including modulation of immune responses that overlap with dysfunctions from immunosuppressive therapy. This patent describes protein-based nanoparticle systems for drug delivery that inherently address side effects like immune dysfunction from chemotherapy. Further, US Patent Published Application 2015/0010631 A1 entitled “Immune-modifying nanoparticles for treatment of inflammatory diseases” adapts protein nanospheres for targeted delivery to mitigate chemotherapy-induced dysfunctions. The motivation arises from improving therapeutic efficacy while minimizing toxicity.

However, careful examination of these two documents shows that they are not relevant to the present technology. The cited reference focuses on nanoparticles for delivering chemotherapy and targeted therapy, not specifically for treating pre-existing dysfunctions arising from chemotherapy or immunosuppressive therapy. The protein nanospheres of the present technology are uniquely formulated to address post-treatment dysfunctions, such as but not limited to immune or other physiological impairments, which are not disclosed or taught by the reference's emphasis on cancer treatment itself. Regarding US 2015/0010631 A1, there is no teaching or suggestion to modify immune-modifying nanoparticles specifically for chemotherapy-induced dysfunctions using our precise protein nanosphere composition and method.

### SUMMARY

In view of the foregoing disadvantages inherent in the known types of treatments to mitigate the damage caused by chemotherapy and/or immunotherapy drugs at least some embodiments of the present technology provides a novel protein nanospheres and method to treat dysfunction from chemotherapy and immunosuppressive therapy, and overcomes one or more of the mentioned disadvantages and drawbacks of the prior art. As such, the general purpose of at least some embodiments of the present technology, which will be described subsequently in greater detail, is to provide a new and novel protein nanospheres and method to treat dysfunction from chemotherapy and immunosuppressive therapy which has all the advantages of the prior art mentioned herein and many novel features that result in a protein nanospheres and method to treat dysfunction from chemotherapy and immunosuppressive therapy which is not anticipated, rendered obvious, suggested, or even implied by the prior art, either alone or in any combination thereof.

According to one aspect, the present technology can include a method of using an albumin nanoparticle suspension containing submicron albumin spheres to mitigate damage to tissue by administration of a multi-valent agent to a subject in need thereof. The method can include the step of administering intravenously a therapeutically effective amount of the albumin nanoparticle suspension containing the submicron albumin spheres to the subject. The albumin spheres can be configured to augment a function or effectiveness of stem cells or precursor cells in vivo to mitigate damage to the tissue caused by treatment to the subject by the multi-valent agent.

According to another aspect, the present technology can include a method of treating one or more side-effects from administration of a multi-valent agent to a patient in need thereof with albumin nanoparticles. The method can include the steps of providing a suspension including fibrinogen-coated albumin nanospheres prepared by coating blank albumin spheres with a solution containing human fibrinogen that is greater than 1.4 mg fibrinogen per mL of the solution. Administering intravenously the suspension to the patient at a concentration of the fibrinogen-coated albumin nanospheres sufficient to augment a function or effectiveness of stem cells or precursor cells in vivo to at least mitigate damage caused by administration of the multi-valent agent to the patient.

According to yet another aspect, the present technology can include a suspension for mitigating damage to tissue caused by administration of a multi-valent agent to a patient. The suspension can include fibrinogen-coated albumin nanospheres prepared by coating blank albumin spheres with a solution containing human fibrinogen that is greater than 1.4 mg fibrinogen per mL of the solution.

In some embodiments, the multi-valent agent can have any one of or any combination of a chemotherapeutic effect and an immunosuppressive effect to the subject or patient.

In some embodiments, the albumin spheres can be configured to have an effect on an oxidative reactivity of the stem cells or precursor cells.

In some embodiments, the albumin spheres can be configured to have an effect on the stem cells or precursor cells as an inducer of anti-oxidants or on an anti-oxidative pathway inside the stem cells or precursor cells.

In some embodiments, the albumin spheres can be configured to maintain a mass of a thymus of the subject or patient, and cells in the thymus.

In some embodiments, the administering of the albumin nanoparticle suspension can be prior to an onset of treatment of the multi-valent drug to the subject or patient.

In some embodiments, the administering of the albumin nanoparticle suspension can be after an onset of treatment of the multi-valent drug to the subject or patient.

In some embodiments, the albumin spheres of the albumin nanoparticle suspension can be bound with fibrinogen molecules to produce Fibrinogen Albumin Spheres (FAS).

In some embodiments, the fibrinogen albumin spheres can be High-Fibrinogen Spheres (HFS) prepared by coating blank albumin spheres with a solution containing human fibrinogen that is greater than 1.4 mg fibrinogen per mL of the solution.

In some embodiments, the solution can contain a concentration of sodium tetradecyl sulphate greater than 5 mg per mL of the solution.

In some embodiments, the sodium tetradecyl sulphate can be configured to keep fibrinogen molecules in a soluble state without precipitation at room temperature.

In some embodiments, the administering of the albumin nanoparticle suspension to the subject or patient can be at a dose greater than 160 mg per kilogram weight of the subject or patient.

In some embodiments, the administering of the albumin nanoparticle suspension to the subject or patient can be at a dose up to 320 mg per kilogram weight of the subject or patient.

There has thus been outlined, rather broadly, features of the present technology in order that the detailed description thereof that follows may be better understood and in order that the present contribution to the art may be better appreciated.

Numerous objects, features and advantages of the present technology will be readily apparent to those of ordinary skill in the art upon a reading of the following detailed description of the present technology, but nonetheless illustrative, embodiments of the present technology when taken in conjunction with the accompanying drawings.

As such, those skilled in the art will appreciate that the conception, upon which this disclosure is based, may readily be utilized as a basis for the designing of other structures, methods and systems for carrying out the several purposes of the present technology. It is, therefore, that the claims be regarded as including such equivalent constructions insofar as they do not depart from the spirit and scope of the present technology.

It is therefore an object of the present technology to provide a new and novel protein nanospheres and method to treat dysfunction from chemotherapy and immunosuppressive therapy that has all of the advantages of the prior art treatments to mitigate the damage caused by chemotherapy and/or immunotherapy drugs and none of the disadvantages.

It is another object of the present technology to provide a new and novel protein nanospheres and method to treat dysfunction from chemotherapy and immunosuppressive therapy that may be easily and efficiently manufactured and marketed.

An even further object of the present technology is to provide a new and novel protein nanospheres and method to treat dysfunction from chemotherapy and immunosuppressive therapy that has a low cost of manufacture with regard to both materials and labor, and which accordingly is then susceptible of low prices of sale to the consuming public, thereby making such protein nanospheres and method to treat dysfunction from chemotherapy and immunosuppressive therapy economically available to the buying public.

Still another object of the present technology is to provide a new protein nanospheres and method to treat dysfunction from chemotherapy and immunosuppressive therapy that provides in the apparatuses and methods of the prior art some of the advantages thereof, while simultaneously overcoming some of the disadvantages normally associated therewith.

These together with other objects of the present technology, along with the various features of novelty that characterize the present technology, are pointed out with particularity in the claims annexed to and forming a part of this disclosure. For a better understanding of the present technology, its operating advantages and the specific objects attained by its uses, reference should be made to the accompanying drawings and descriptive matter in which there are illustrated embodiments of the present technology. Whilst multiple objects of the present technology have been identified herein, it will be understood that the claimed present technology is not limited to meeting most or all of the objects identified and that some embodiments of the present technology may meet only one such object or none at all.

### BRIEF DESCRIPTION OF THE DRAWINGS

The present technology will be better understood and objects other than those set forth above will become apparent when consideration is given to the following detailed description thereof. Such description makes reference to the annexed drawings wherein:

FIG. 1 is a graphical representation of the size distribution of spheres in a suspension of FAS in accordance with the principles of the present technology.

FIG. 2 is a graphical representation of the percent (%) change in body weight of different dosing groups.

FIG. 3 is a graphical representation of the thymus and spleen weights (mg) on Day 12.

FIG. 4 is a graphical representation of the thymus and spleen weights in the 4 groups on Day 17.

FIG. 5 is a graphical representation of the comparison of the white blood cell (WBC) counts of various groups on Day 12.

FIG. 6 is a graphical representation of the white blood cell (WBC) counts including the differentials of various groups on Day 17.

FIG. 7 is a graphical representation of the red blood cell (RBC) concentrations on Day 12.

FIG. 8 is a graphical representation of the red blood cell (RBC) concentrations on Day 17.

### DETAILED DESCRIPTION OF THE EMBODIMENTS

While the known treatments may fulfill their respective, particular objectives and requirements, the aforementioned devices or systems do not describe a protein nanospheres and method to treat dysfunction from chemotherapy and immunosuppressive therapy that allows mobilizing a patient's own stem cells and other mechanisms to mitigate harm caused by agents with multiple functions or targets, including those used in chemotherapy, immunosuppressive therapy and radiation therapy.

A need exists for a new and novel protein nanospheres and method to treat dysfunction from chemotherapy and immunosuppressive therapy that can be used for mobilizing a patient's own stem cells and other mechanisms to mitigate harm caused by agents with multiple functions or targets, including those used in chemotherapy, immunosuppressive therapy and radiation therapy. In this regard, the present technology substantially fulfills this need. In this respect, the protein nanospheres and method to treat dysfunction from chemotherapy and immunosuppressive therapy according to the present technology substantially departs from the conventional concepts and designs of the prior art, and in doing so provides an apparatus primarily developed for the purpose of mobilizing a patient's own stem cells and other mechanisms to mitigate harm caused by agents with multiple functions or targets, including those used in chemotherapy, immunosuppressive therapy and radiation therapy.

In the following description, for purposes of explanation and not limitation, specific details are set forth, such as particular embodiments, procedures, techniques, etc. in order to provide a thorough understanding of the present technology. However, it will be apparent to one skilled in the art that the present technology may be practiced in other embodiments that depart from these specific details.

This patent application discloses a product and a method to treat the harm caused by “multi-valent agents” (also called multi-agent in this application.) Certain drugs can serve as more than one class of therapeutic agent, such as serving as chemotherapeutic agents as well as immunosuppressive agents. They target more than one tissue or entity such as cancer cells, overactive immune cells or infectious agents. Therefore, it is hard to expect one mitigation agent (such as proposed in this patent application) to be effective in reversing or even healing the harm done to more-than-one-type-of-tissue. When two target tissues are identified, these “treatment drugs” (for the diseases) are often called dual-use agents. When more than two targets are known or suspected, they can be called “multi-valent agents” or “multi-agents.” Although typically administered to different patient populations (e.g. one population with cancer, the other with autoimmune or related immune disease) some such drugs can be used in the same patient who has more than one healthy problem that can be “treated” with the same multiagent. However, the multi-agent can by itself cause a different set of harm to the patient. The mechanisms of action of such multi-agents can be complex and often intertwined with the body's homeostatic mechanisms. It is the intention of this description to explain why protein nanoparticles of the present technology is uniquely useful toward the mitigation of the harm caused by multi-agents while allowing the multi-agents to be effective in their healing power.

### Part I: Cancer-Related and Immune-Related Problems

Although most people consider cancer to be a distinctly different disease from immune-related diseases, recent studies revealed that in many situations they are “connected” because the same set of cells are used to fight against both conditions. In Part II and III (Cellular Immunity and Dose-dependent Chemotherapy) we will further explore how the body regulated these activities and how “medications” can be used to advantage for the patient.

In this patent application, we make a distinction between the term “immune-compromised” versus “immune-suppressed.”

The Immune-compromised (or immunocompromised) condition can be caused by a variety of factors, which are typically the result of other conditions and are not the intention of a therapy. One example of an “immunocompromised condition” is an infection by the HIV (Human Immunodeficiency Virus) which will render the patients susceptible to infectious agents, plus the development of cancer cells in the patient. In contrast, immunosuppression is administered to patients with over-active immune systems for example autoimmune diseases, where the therapy is purposefully aimed at suppressing the overactive function or the concentration of immune cells, so that the “immune status” of the patient can be more normal.

To clarify our discussion below, we intend to point out that there are at least 3 levels of patient condition:
(a) the disease itself, e.g. patient comes in with a diagnosis of having an auto-immune disease;
(b) a conventional method of treatment is administered. We intend to call this drug or therapy the “treatment drug”- and if this drug has multiple functions, we intend to call it a multi-valent or multi-agent. One example will be CTX; and
(c) a “mitigation agent” such as disclosed here which aims at reducing or reversing the harm done by the “treatment agent” (which is given for the treatment for the disease.)

The ideal mitigation agent will reverse some or all of the harm done by the “treatment agent” without making the disease condition worse and without negatively impacting the effectiveness of the “treatment agent.” One such mitigation or mitigative agent will be Fibrinogen-coated Albumin Spheres (FAS). Another way of stating this is that our “mitigative agent” aims at reducing or reversing the “side-effect” of the “treatment agent.”

There are many kinds of Immune-compromised patients as discussed in Hematology.org: “Treatment of COVID-19 in Immunocompromised Patients with Hematologic Conditions” Sep. 13, 2022. These patients include:
a. Active treatment for solid tumors or hematologic malignancies
b. Receipt of solid-organ transplants and on immunosuppressive therapy
c. Receipt of chimeric antigen receptor T-cell (CAR-T) therapies or hematopoietic cell transplantation (HCT) within two years of transplantation, or still requiring immunosuppressive therapy
d. Moderate or severe primary immunodeficiency disorders (e.g., DiGeorge syndrome, Wiskott-Aldrich syndrome, severe combined immunodeficiencies, etc.)
e. Advanced or untreated HIV infection (most significantly, CD4 cell counts <200/mm3, history of an AIDS-defining illness without immune reconstitution, or other clinical manifestations of HIV) f. Active treatment with high-dose corticosteroids (i.e., >20 mg of prednisone or equivalent per day administered for 2 or more weeks), alkylating agents, antimetabolites, transplant-related immunosuppressive drugs, cancer chemotherapeutic agents classified as severely immunosuppressive, tumor necrosis factor blockers, and other biologic agents that are immunosuppressive or immunomodulatory

Unfortunately for these immunocompromised patients, they have a high rate of cancer occurrence. See: Cancer Therapy Advisor (author Jessica Nye, Apr. 4, 2022): reporting on a National Comprehensive Cancer Network 2022 Annual Conference Report that the rate of new cancer in the Tumor Necrosis Factor Inhibitor (TNF-i) group was 8.8%, in the transplant (solid organ or hematopoietic stem cell transplant) group was 11.5%, and 14.3% in the Primary/Secondary Immunodeficiency Disease Group. These rates are almost double that of baseline rates in comparable groups without the immunocompromised condition. Among the new cancers that they may develop: gastrointestinal (14.35), hematopoietic (12.4%) and skin (12.4%) cancers are most common. Among patients receiving TNF-I, common cancers are the reproductive system (17.4%) and breast (5.9%) cancers. These cancers will be treated with chemotherapy or radiation. However, the chemotherapy or radiotherapy may not be tolerated by these immunocompromised patients as compared to other cancer patients who are not immunocompromised. Therefore, a treatment such as disclosed here that will mitigate the harm of chemotherapy to these patients will be very important, yet non-obvious from the prior arts as to how this can be achieved.

For patients who are already immunocompromised, the treatment for cancer can further weaken their immune system. Therefore, there is an urgent need to augment their immune system before further damage is done. These patients suffer from the combined effects of the “immunocompromised state” and their cancerous state. In addition, they will suffer from the side effects of their “cancer treatment” (chemotherapy or radiation therapy) which can easily bring about the demise of the patient.

In term of “immunosuppressed” patients, their immune system was originally “over-active” (e.g. making too much of useless, unneeded or unwanted antibodies, or immune cells.) Or these patients produce harmful, inappropriate and “bad” antibodies or “bad” immune cells that attack the healthy cells of the patient. The immunosuppressive therapy can bring out at least three further complications: (a) the patient's immune system after treatment is still relatively over-active because the immunosuppression is not adequate; (b) their immune system is near normal but unstable even though the immunosuppressive therapy is appropriate at the start of the suppressive therapy; (c) their immune system is rendered weak due to the fact that the immunosuppressive therapy is too strong for them. These patients then need delicate treatment whether they develop “new” cancer, or no cancer. One approach is to do two things at the same time: (a) first, augment the “good side” of the immune system: by utilizing the present technology to increase the production of cells that can produce the “good” antibodies or immune cells (e.g. those fighting infections), while (b) at the same time, allow the treatment agent (such as CTX) to suppress the “bad” immunoglobulins or cells, such as those attacking the healthy cells of the patients. This approach has the advantage of allowing a higher immunosuppressive dose because the good-antibody-producing ability or “good immune cells” have been protected by the present technology.

There are a lot of patients in the USA who are immunocompromised or immunosuppressed who later developed cancers. The following table (Table 2) listed the 10 most common types of cancer which arise in patients receiving a certain type of immunosuppressive therapy (2nd column), who are then treated with certain therapies, including chemotherapy (3rd column). The number of such patients (4th column) is estimated from a search of various scientific and medical papers including those from the American Cancer Society and the National Cancer Institute.

TABLE 2
Types of cancers found in patients receiving immunosuppressive
therapy and the treatment for their cancers.

| | | | Number of |
|---|---|---|---|
| | Immunosuppressive | | Cases in |
| Type of Cancer | Drug/Therapy | Treatment/Chemotherapy | the USA |
| Non-Hodgkin | Tumor Necrosis Factor | Chemotherapy (CHOP), | ~77,240 |
| Lymphoma (NHL) | (TNF) inhibitors, | Rituximab | |
| | Calcineurin inhibitors | | |
| Kaposi's Sarcoma | HIV/AIDS, | Antiretroviral therapy | ~6,000 |
| | Immunosuppressive therapy | (ART), Chemotherapy | |
| Lung Cancer | Immunosuppressive drugs | Surgery, Chemotherapy, | ~228,820 |
| | post-transplant | Immunotherapy | |
| Kidney Cancer | Immunosuppressive drugs | Surgery, Targeted therapy, | ~73,750 |
| | post-transplant | Immunotherapy | |
| Liver Cancer | Hepatitis B/C infection, | Surgery, Chemotherapy, | ~42,810 |
| | Immunosuppressive drugs | Targeted therapy | |
| Skin Cancer | Immunosuppressive drugs | Surgery, Immunotherapy, | ~106,110 |
| (Melanoma) | post-transplant | Targeted therapy | |
| Gastrointestinal | Immunosuppressive drugs | Surgery, Chemotherapy, | ~145,600 |
| Cancer | post-transplant | Targeted therapy | |
| Breast Cancer | Tumor Necrosis Factor | Surgery, Chemotherapy, | ~281,550 |
| | (TNF) inhibitors | Radiation therapy | |
| Hematopoietic | Immunosuppressive drugs | Chemotherapy, Stem cell | ~60,530 |
| Cancer | post-transplant | transplant | |
| Cervical Cancer | Human Papillomavirus | Surgery, Chemotherapy, | ~13,800 |
| | (HPV) infection, | Radiation therapy | |
| | Immunosuppressive drugs | | |

In this patent application and in the exemplary, we focus on one particular chemotherapeutic drug which also has immunosuppressive effects, i.e. cyclophosphamide (CTX). However, the benefit of the present technology is not limited to the use of CTX but is widely applicable to similar drugs or treatment modalities with more than one treatment target, including unwanted cells causing a variety of health issues. In addition, in many scientific papers, the authors do not distinguish or make a distinction whether CTX is used mainly as a chemotherapeutic drug or as an immunosuppressive agent. Furthermore, CTX is used in a combination of many other drugs. Therefore, in this application, when we mention CTX as a “chemotherapeutic agent” we do not intend to say that it is not an immunosuppressive drug: the effect of this drug will do whatever it can do to the patient. Health-providers may find it helpful to administer drugs that have dual-functions (or more than 2 functions) when the patient can benefit from more than one mechanism of action from the drug.

With specific attention to CTX, Table 3 below lists the 5 most common cancers that arise in patients whose treatment includes CTX. It can be seen that although some companion drugs (used in combination with CTX) are listed as immunosuppressive drugs, they are also included as chemotherapeutic agents

TABLE 3
The five most common cancers that arise in patients receiving
various drugs including CTX, either as the immunosuppressive
drug or having CTX mainly as the chemotherapeutic drug

| | Immunosuppressive | |
|---|---|---|
| Type of Cancer | Drug | Chemotherapy Regimen |
| Non-Hodgkin | Rituximab, | Cyclophosphamide, |
| Lymphoma (NHL) | Methotrexate | Doxorubicin, Vincristine, |
| | | Prednisone (CHOP) |
| Breast Cancer | Doxorubicin, | Cyclophosphamide, |
| | Paclitaxel | Doxorubicin (AC) |
| Acute Myeloid | Cytarabine, | Cyclophosphamide, |
| Leukemia (AML) | Daunorubicin | Cytarabine, Daunorubicin |
| Multiple Myeloma | Bortezomib, | Cyclophosphamide, |
| | Lenalidomide | Bortezomib, Dexamethasone |
| | | (CyBorD) |
| Ovarian Cancer | Carboplatin, | Cyclophosphamide, |
| | Paclitaxel | Carboplatin, Paclitaxel |

A comparison of Table 2 and Table 3 will show that two types of cancers are very often seen in patients receiving immunotherapy or chemotherapy, including CTX, even though CTX is not listed in Table 2 specifically. These cancers are: Non-Hodgkin Lymphoma (NHL) and breast cancer. Therefore, we will investigate if (a) our present technology is beneficial in the mitigation of the toxic effects of CTX, with or without the presence of cancer, (b) the administration of FPS does not decrease the effectiveness of CTX in killing cancer cells, including the breast cancer and NHL, (c) the administration of FPS does not increase the survival or proliferation of cancer cells during or after CTX treatment or the use of other conventional therapies for the cancer.

### Part II: Cellular Immunity Importance of Regulatory T Cells (Treg)

“Immunity” is a term generally understood to mean “defense of the body against infectious agents.” However, the same system is used against the “rise of cancers within the body.” Here we will discuss how the body tries to maintain a “balance” so that neither infectious agents (bacteria or virus or any other agents) nor cancer cells can gain the upper hand against the homeostasis (“well-being”) of the body.

There are mainly two systems of “immunity” in the body: (a) the soluble system—which is the antibody system. Although antibodies are produced by cells, the antibodies are soluble proteins which allow them to reach almost everywhere in the body. (b) The other system is the cellular system—mainly the “T cell” system. We will focus on the Cellular Immune system here because it is this system that the body uses mainly to fight against cancer. The two systems (the antibodies and the cells) often work together for “mutual” support.

There are many types of T cells, e.g. Effector T cells (Tef); Helper T cells (Th); Regulatory T cells (Treg), etc. They often upregulate or downregulate in their concentration and their function in the body, depending on what is needed to maintain good health. They interact with each other and therefore can be confusing so to “who is doing what to whom under what condition.” To simply our discussion, this author will use several “levels” of control (or regulation) so that the complicated description in scientific papers with respect to cellular immunity can be understood easily.
a) Level 1: Here we discuss mainly the Effector T cells (Tef). The condition is this: some bacteria invade the body. The body had previously been in contact with this kind of infectious agent. The “memory cells” immediately go to work and allow a large quantity of antibodies to be produced. (This is the rationale for “vaccination”). However, antibodies are of several types: some are called “neutralizing antibodies” because they “alone” can neutralize the bug (with help from some other cells). Others are called “non-neutralizing antibodies”—they merely tag onto the bacteria without killing the bacteria. The “tagging of the bacteria” will send a stronger signal for other cells to come, which are capable of killing and eating the bacteria. Although the situation can be complicated, we will simplify by grouping these Level 1 cells under the umbrella of the Tef
b) Level 2: Sometimes Tef is not strong enough. So, the body calls upon another type of T cells. We will call these the T helper cells (Th.) Th will help the Tef to accomplish the task of wiping out the entire invasion.
c) Level 3: When the Th and Tef have done their job, there is no more bacteria to be killed. Therefore, the body needs to remove the existing Th and Tef in the blood (or at least stop making more of them.) These Th and Tef need to be regulated because “concentrations higher than normal” (and for long durations) may end up harming the body's own cells (a condition called “hyperactive immune system”—leading to autoimmune diseases.) Here, the T Regulatory cells (Tref) come into play. They “regulate” the Th and Tef function and numbers. It is recognized that Tref may need “activation” so that they will not randomly stop the “wrong” kind of Th or Tef which may still be needed elsewhere to kill a different kind of infection.
d) Level 4: Given the importance of Treg in stopping waste-of-resources when infection is controlled and in the prevention of autoimmune diseases, one would think that in general, a high level of Treg (compared to the level in a normal, non-infectious state) is good. However, that is not true. It has been found that a high level of Treg is bad for the body in terms of controlling the development of cancer. In fact, Treg has been isolated from tumor masses where the Treg are hiding and participating in the growth of tumors against the surveillance system of the body. Some scientists will call this kind of surveillance the “anti-cancer” regiment of the body. However, we will try to avoid the term “anti”, because the word “anti” needs to be qualified every time to specify what is being “worked against.” Even with such specification of what the regiment is working against, after a few rounds, nobody can be sure whether it is good or bad for the patient. For example, a statement like this can be totally confusing: “W” is anti-X which is effective in suppressing Y which as usually an “Anti-Z” agent. Therefore, this author will discuss mainly “positive effects” (for the body) rather than “anti-effects” against a particular “level” of interaction.
e) Level 5: Given the observation that most (healthy) people do not have severe infections (nor hyperreactive immune system) and they do not have cancer, there must be another level which we call Level 5 where the Treg is neither too high (to be down-regulated if it is so) nor too low (to be up-regulated if it is too low.) There is little scientific study in this Level 5 but we hypothesize that it must exist. The treatment agent (e.g. CTX) used to control autoimmune diseases/cancer will likely affect this level of cell involvement as well as the previous levels of control (Level 1 to 4).
f) Level 6: Now we talk about the muti-valent treatment agent. The effect of multi-agents can be complicated. It needs to work on the remission of cancer while at the same time having an effect on the immune system. Or, if the patient is having an autoimmune problem the muti-agent needs to control the overactive immune system without causing the development of cancer. The agility of this “treatment agent” (a single-effect agent or a multi-agent) can be compared to a “ping-pong” match. The ping-pong table has two sides. If the ball is landing on the “east-side” of the table, “somebody” needs to hit it back to the “west-side” of the table. But if it lands too “hard” on the “west-side” of the table, and nobody can hit the ball back; the game is finished.
g) Level 7: Now we talk about the “Mitigation Agent.” Given the fact that the multi-agent can have toxic effect on the body as well as the cancer cells as well as immune cells, the mitigation agent (e.g. FAS) must work “in harmony” with everybody involved. Hence the detailed description in this disclosure.

The applicant of this patent application wants to stress that, in spite of the simplification listed above, in reality the immune system is very complex. For example, for Helper T cells alone, in the book called Molecular Biology of the Cell (4th edition) the authors (Alberts et al) described some activities and interactions of Helper T cells and lymphocyte activation. They said,
“Helper T cells are arguably the most important cells in adaptive immunity, as they are required for almost all adaptive immune responses. They not only help activate B cells to secrete antibodies and macrophages to destroy ingested microbes, but they also help activate cytotoxic T cells to kill infected target cells. As dramatically demonstrated in AIDS patients, without helper T cells we cannot defend ourselves even against many microbes that are normally harmless.”

The authors continued:
“Helper T cells themselves, however, can only function when activated to become effector cells. They are activated on the surface of antigen-presenting cells, which mature during the innate immune responses triggered by an infection. The innate responses also dictate what kind of effector cell a helper T cell will develop into and thereby determine the nature of the adaptive immune response elicited.”

Given the complexity of the interactions between various systems and cells and treatment agents, it may be helpful to have a simple table which is intuitive to people interested in grasping all the facts. Therefore, the author of this disclosure has created the following table which will focus on the effect of high versus low Treg toward the control of bad situations such as cancer formation on the one hand and autoimmune disease creation on the other hand. Table 4 below shows what is a “good” condition for the body and what is a “bad” condition for the body given that the two diseases are both bad for the body.

TABLE 4
Intuitive way to understand the effect of multi-agents such
as CTX on different kinds of disease conditions or patients

| | | AUTOIMMUNE |
|---|---|---|
| | CANCER DEVELOPMENT | DEVELOPMENT |
| HIGH concentrations of | BAD for the body because High | GOOD for the body because |
| Treg or their function | Treg weakens tumor- | High Treg controls and reduces |
| | surveillance system | Th and Tef activity |
| LOW concentrations of | GOOD for the body because | BAD for the body because low |
| Treg or their function | low Treg promotes suppression | Treg allows an overact immune |
| | of cancer development | system to develop |

Therefore, a “good” chemotherapy agent or a “good” immune-suppressive agent (or an agent good for both systems) must be able to do both: (a) being effective against a High Treg concentration (i.e. will bring it down) if the patient has a cancer problem, while (b) being effective against a low Treg concentration (by raising its level) if a patient has an overactive immune system. The following Part III will describe why this is the case.

### Part III: Dose-Dependent Chemotherapy

Now we talk about the treatment modalities or treatment agents. Most people understand that chemotherapeutic agents are used to kill cancer cells; but they also do some damage to the healthy cells. Therefore, the conventional approach is to use as high a dose of the chemotherapeutic agents (to kill as many cancer cells) as possible until the patient cannot tolerate the side-effects (bad effects on the healthy cells or organs) anymore. Reality, however, can be more complicated.

Various research scientists have confirmed a curious and counter-intuitive result: that a low dose of a chemotherapeutic agent, namely CTX is effective against certain cancer cells (e.g. hepatocellular carcinoma, HCC) while high doses of CTX are not effective. How come? It turns out that the agent CTX, when used in a low dose, is primarily killing Treg cells and not directly killing the tumor cells. Reducing the unhelpful high concentration of the Treg in the body will then allow the body's other systems to kill the cancer cells effectively. At the same time, the data show that a high dose of CTX is not good to the body because a high dose of CTX kills all the T cells (pan-cytotoxic) leaving the cancer cells free to grow.

One specific publication is worth mentioning. There is an article titled “Low-dose Cyclophosphamide Treatment Impairs Regulatory T Cells and Unmasks AFP (alpha fetal protein)—specific CD4+ T-Cell Responses in Patients with Advanced HCC (Hepatocellular Carcinoma)” authored by Greten et al in Journal of Immunotherapy 33(2):p2l 1-218. The authors published data on how low doses of CTX will “unmask” other T-cells which will kill HCC, while the high doses of CTX will not.

In this patent application, we will use the term “low dose” or “high dose” the same way as used by the authors in each of the different publication, because of the different models that they use for their studies. We do not use an absolute concentrations as indication whether it is a “high” or “low” dose because each paper tends to disclose a slightly different dose for their study.

The publication by Greten et al explained many previously unexplained and perplexing findings in Cancer-Vaccination studies. Cancer-Vaccination is the idea of treating cancer-derived material as if they are infectious agents. The question is: If cancer cells are in some way “foreign” or “out of control” to the body, will injection of cancer-derived-material result in the building up of “immunity against cancers” the same way injection of bacteria-derived-material will build up immunity against bacterial invasion? To date, there is not much success for this approach. The reason is: the activity of Treg must be regulated first. An article was published in 1990, authored by Hoon et al, titled “Suppressor Cell Activity in A Randomized Trial of Patients Receiving Active Specific Immunotherapy with Melanoma Cell Vaccine and Low Dosages of Cyclophosphamide” in Cancer Res. 1990; 50(17):5358-64. (The authors named certain “Suppressor Cells” which are probably what we now call Treg.) They found that the administration of a low dose of CTX will reduce the concentration of their “Suppressor Cells” (which are actually Treg cells) but the low dose CTX should be given 3 days before the administration of the “vaccine” (which are cell-surface melanoma-associated antigens.) We now understand why-because these cancer patients already have high concentration of Treg, which will need to be reduced first, by a low-dose treatment of CTX; then the administration of the vaccine will be more effective. Hence, the health-provider must pay attention to the vital role exerted by Treg, and particularly its concentration and function in the cancer-occupying body.

### Part IV: Mitigation of The Toxicity of Multi-Agents by the Use of Dose-Dependent Mitigation Agents

With regards to the present technology, it is beneficial to mention that the term “multi-agent” as used in this application refers to the cancer-treatment agent or the auto-immune-treatment agent, of which CTX is only one example here. The term “Mitigation Agent(s)” refers to protein nanoparticles including Fibrinogen-coated Albumin Spheres (FAS) or the High-Fibrinogen Spheres (HFS) which will be used to remedy the harm caused by the multi-agent, in the presence or absence of specific diseases.

While it has been demonstrated that high-doses of CTX can have effects different from low-doses of CTX, it is hypothesized here that FAS as a Mitigation Agent may also have differentiating effects when used in high doses versus low doses. In most medical language, the word “dose” refers to the “mass of the medicine given to the patient.” The mass can be in solid weight (e.g. milligram per pill) or the mass in suspension (e.g. 8 mg of spheres per mL)—the dose being expressed as 1 mL of suspension per kg; or 8 mg sphere per kg weight of the patient. However, in this application, we also include another definition of “dose” which is the “amount of fibrinogen per sphere”—fibrinogen being the “active substance” for the “homing device.” It should be pointed out the “soluble fibrinogen” will not have the same effect as “fibrinogen attached to the sphere. Also, soluble albumin or blank albumin spheres will not have the same medical effect as “albumin spheres with attached fibrinogen, or coated with fibrinogen molecules.” This is because the sphere is an integral part of the present technology. The sphere provides the “mass” or “body” for the “combination” consisting of a protein nanosphere plus an appropriate mass of fibrinogen molecules attached to the surface of that sphere-only the “combination” will work.

We will point out in the following discussion (a) the quantity of spheres to be administered to a patient (e.g. 8 mg of FAS per kg weight of the patient). In this case, the FAS was prepared by the standard method as disclosed in previous Yen patents, having about 5% w/w, i.e. about 5 mg of fibrinogen molecules attached to each 100 mg of FAS spheres. (b) However, the High-Fibrinogen Sphere (HFS) preparations will have a higher concentration of fibrinogen per sphere, i.e. having higher than 5 mg of fibrinogen per 100 mg of HFS spheres. This HFS product is new in that previous attempts to add a higher concentration of fibrinogen to a standard volume of blank spheres have failed to produce useful spheres—in fact some batches will fail after a short period of storage in room temperature. The method to produce useful and stable HFS will be described below. This product and its method of production is novel and non-obvious.

Referring to the statement above that: “it is hypothesized here that FAS as a Mitigation Agent may also have differentiating effects when used in high doses versus low doses”—we mean both (a) the dose to be administered to the patient (in terms of mg spheres per kg weight of the patient, regardless of which kind of spheres, Standard or High Fibrinogen Spheres) as well as (b) the kind of spheres to be used (Standard FAS versus HFS).

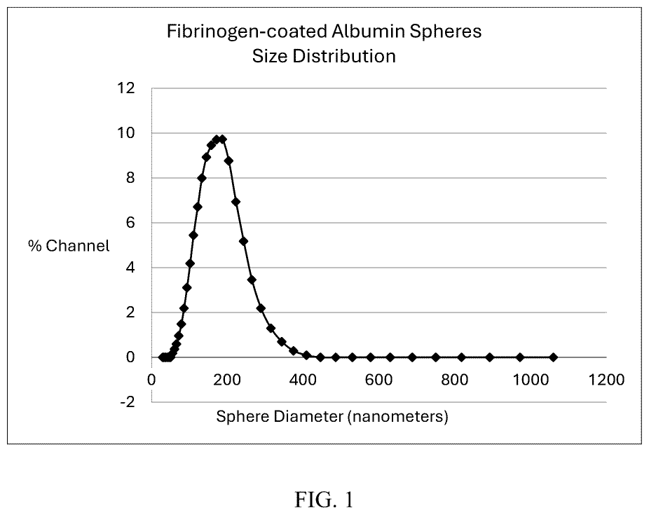

This is a reasonable hypothesis because FAS is a biological product. In contrast, the term “drugs” as defined by the Food and Drug Administration are “single chemical entities”: i.e. all the molecules in the dose of treatment shall be chemically identical. However, FAS is a biological product: it can be comprised of a population of “almost identical entities.” For example, in the production of FAS, a standard solution of fibrinogen molecules is added to “blank albumin spheres” to coat them. Even though the “average number of fibrinogen molecules coating per sphere” is very consistent from batch to batch; it can be understood readily that the exact number of “fibrinogen molecules per sphere” will vary slightly from sphere to sphere—it is not identical (even though the range is a very narrow range). This can be simply due to the fact that the sizes of the blank spheres fall within a narrow range (with a median diameter of 158 nanometer) but they are not 100% 158 nanometer in diameter. FIG. 1 showed that a typical suspension of FAS contains the following parameter: no spheres larger than 409 nM; less than 1% larger than 344 nM; median diameter is 158 nM; less than 1% smaller than 66 nM; none less than 51 nM. Even though a standard solution of Fibrinogen is used to coat the suspension of spheres, it is clear that the number of fibrinogen molecules per sphere will vary between different spheres of different sizes; and the density of Fibrinogen per surface area may also vary depending on the surface area of that particular single sphere.

One may hypothesize that (a) only the “spheres with the highest Fibrinogen concentration per sphere” (High-Fibrinogen-content Spheres) are best suited to mitigate the harm caused by a certain dose of CTX, (b) but only 1% of the population of the FAS preparation are “High-Fibrinogen-content Spheres”-therefore, a larger dose of FAS is needed, e.g. a dose of 80 mg of FAS per kg is needed, which will then contain enough High-Fibrinogen-content Spheres to be effective in the mitigation of the harm caused by CTX. However, in the HFS preparation, it is possible that 10% of the spheres in the suspension will have spheres which have High-Fibrinogen-content. Therefore, by using HFS preparations, a dose of 8 mg of HFS suspension per kg is enough to benefit the patient.

It can be understood readily that a high dose of CTX may have an effect quite different from a low dose of CTX on the thymus (which is needed to educate the naïve T cells) and its content of T cells. Therefore, in terms of the mechanism of action whereby FAS or HFS can mitigate the harm done by CTX, it may be further hypothesized that (a) a high dose of FAS can mobilize all kinds of stem cells (e.g. from the bone marrow) needed to repair the pan-cytotoxic effect of a high dose of CTX on the T cell-producing organs and all the T-cells that originate from these organs; while (b) a low dose of FAS may be enough to mobilize a lower concentration or an easier-to-mobilize population of stem cells from their site of origin (e.g. the bone marrow, but not limited to the bone marrow) to go to the Thymus (or other T cell producing organs) to heal the harm there which was done by a low dose of CTX (but not a high dose of CTX.) Therefore, in a situation where a high dose of CTX must be used to control the patient's disease condition (e.g. to control a severely auto-immune patient's condition) a high dose of FAS must be used.

Again, the term FAS is used in the above paragraph; the statements apply equally well to the use of HFS with respect to its effectiveness in high versus low doses of CTX administered to the patient.

The experiments to be described below used a high dose of FAS to demonstrate the effect of such a high dose of FAS on maintaining the mass of the thymus (and the cells in it) which would be otherwise destroyed by a high dose of CTX. Further experiments will provide data to show the effects of a low dose of FAS, and whether such low doses of FAS have slightly different effects compared to high doses of FAS. The exact mechanism of action relating the beneficial effects of FAS in the mitigation of the harm caused by high-doses of CTX has yet to be studied: however, if a high dose of FAS will work on the concentration on Treg (or cells under the control of Treg) regardless of the dose of CTX to be used, it will reduce the level of complexity (such as the 7 levels of complexity described above) and will greatly simplify the regiments of treatment to be used for patients with cancer or patients with autoimmune diseases, and particularly for patients with both diseases.

The safety of FAS has been evaluated in a cGLP (Good Laboratory Practice) study. In animal studies involving the administration of fluids, there is a limit as to how much fluid an animal can tolerate before the animal will suffer from fluid overload, leading to undesirable conditions such as congestive heart failure. Typically, animals can tolerate 10 mL per kg weight, above which the animal will show difficulty in breathing (congestion in the lung) and other unhealthy signs and symptoms. To reduce the risk associated with fluid overload, one approach is to provide a longer time for the infusion of the fluid, so that the animal can remove excessive fluid in the urine. A high dose of FAS has been evaluated, using 20 mL (equal to 160 mg of spheres) per kg weight of the animal (rats.) Instead of a bolus, the entire volume is infused slowly at a rate of 20 mL per hour. This means for a male rat weighing 400 gram, the volume of FAS to be infused will be 20 ml×0.4 kg or 8 mL, which will be infused at a rate of 20 mL per hour, or over 0.4 hour (24 min.) By comparison, a rat weighing 300 gram will have a total volume of 6 mL infused over a period of 0.3 hour (18 min.) The data showed that there were no clinical signs (of ill effects). There were also no abnormalities in any of the laboratory values and no abnormalities in any of the tissues and organs studied.

The safety of HFS will need to be studied as thoroughly as the FAS preparation. We expect that HFS will have a safety profile equal or better than FAS. The fact that the FAS cGLP study showed safety even with the highest dose evaluated at 160 mg per kg indicated that even higher doses can be used (provided the infusion rate is safe). We believe that both FAS and HFS can be safely administered at doses of 40 ml (equal to 320 mg spheres) per kg or even higher.

Part V: Other Medical Uses for Cyclophosphamide which May Cause Toxicity that Could be Mitigated by FAS

It should be noted that in this present disclosure, we focus in the exemplary on one drug which is CTX. However, the benefit of the present technology is not limited to that of CTX but is widely applicable to any drug with a chemotherapeutic effect and/or immunosuppressive effect (including immunocompromising effects.)

In terms of the application of CTX to patients, there is an excellent description of CTX and its side effects in “General Principles of the Use of Cyclophosphamide in Rheumatic Diseases” authored by McCune and Clowse in UpToDate. The link is: www.uptodate.com/contents/general-principles-of-the-use-of-cyclophosphamide-in-rheumatic-diseases.

The Introduction in that paper states: “Cyclophosphamide (CYC), an alkylating agent, is one of the most potent immunosuppressive therapies available. It has been used extensively to treat severe manifestations of a variety of autoimmune and inflammatory diseases. Examples include organ-threatening manifestations of rheumatic diseases such as systemic lupus erythematosus (SLE), granulomatosis with polyangiitis (GPA), microscopic polyangiitis (MPA), polyarteritis nodosa, eosinophilic granulomatosis with polyangiitis (EGPA; Churg-Strauss syndrome), Behget syndrome, primary angiitis of the central nervous system, and isolated vasculitic neuropathy.”

The authors further stated: “It is a prodrug that is converted to its active form in the liver and, to a lesser extent, in other organs. It can be administered either orally or intravenously. Oral administration usually corresponds to daily dosing and intravenous use to intermittent dosing (e.g., every two to four weeks), but some exceptions exist. For example, extremely ill patients who are unable to ingest medications orally may rarely receive daily doses of CYC via the intravenous route at the same doses they would otherwise receive orally.

Although very effective, CYC has the potential for devastating toxicity both in the short and long term (even after the medication has been stopped). Concerns about such drug toxicity, especially malignancy, have restricted its use to patients with the most severe disease, or patients who are either intolerant of or unable to adhere to less toxic orally administered immunosuppressive drugs (e.g., mycophenolate mofetil). Newer, short-term protocols using significantly reduced cumulative doses of CYC have reduced, but not eliminated, associated risks.”

In terms of the use of CTX in immune-overactive patients, table 5 below listed the top five diseases that are often treated with CTX, their dosing regiments and major complications from the treatment.

TABLE 5
Diseases treated with CTX and their complications

| Disease | Dose and Regimen | Complications |
|---|---|---|
| Systemic Lupus | 500-1000 mg/m² IV every | Infections, Hemorrhagic |
| Erythematosus (SLE) | month for 6 months, then | cystitis, Bone marrow |
| | every 3 months for 2 years | suppression |
| Granulomatosis with | 2 mg/kg/day orally for 3-6 | Infections, Hemorrhagic |
| Polyangiitis (GPA) | months | cystitis, Bone marrow |
| | | suppression |
| Microscopic Polyangiitis | 2 mg/kg/day orally for 3-6 | Infections, Hemorrhagic |
| (MPA) | months | cystitis, Bone marrow |
| | | suppression |
| Rheumatoid Arthritis (RA) | 1-2 mg/kg/day orally for 8-12 | Infections, Hemorrhagic |
| | weeks | cystitis, Bone marrow |
| | | suppression |
| Systemic Sclerosis (SSc) | 500-750 mg/m² IV every | Infections, Hemorrhagic |
| | month for 6 months | cystitis, Bone marrow |
| | | suppression |

Table 6 below showed the incidence and prevalence of these diseases and the cost of treatment per patient.

TABLE 6
Incidence and prevalence of major immune-related diseases in the USA

| | Number of New | | |
|---|---|---|---|
| | Cases in the USA | Prevalence in | Cost of Treatment per |
| Disease | (per year) | the USA | Patient |
| Systemic Lupus | ~16,000 | ~204,000 | $28,298-$68,260 per year |
| Erythematosus (SLE) | | | |
| Granulomatosis with | ~1,300 | ~3 per 100,000 | Varies, often includes high- |
| Polyangiitis (GPA) | | | dose corticosteroids and |
| | | | immunosuppressive drugs |
| Microscopic | ~1,500 | ~1.5 per | Varies, often includes high- |
| Polyangiitis (MPA) | | 100,0007 | dose corticosteroids and |
| | | | immunosuppressive drugs |
| Rheumatoid Arthritis | ~1.5 million | ~1% of the | $10,000-$30,000 per year |
| (RA) | | population | |
| Systemic Sclerosis | ~20,000 | ~75,000 | $20,000-$50,000 per year |
| (SSc) | | | |

The number of new cases and the cost of treatment listed above can be located in the public domain such as publications from the Center for Disease Control of the USA government and other scientific publications.

As discussed above, the effect of CTX on the immune system is complicated. There is an article titled “The Effect of Cyclophosphamide on the Immune System: Implications for Clinical Cancer Therapy” authored by Ahlmann et al, published in Cancer Chemotherapy and Pharmacology Vol 78, pages 661-671, 2016. The authors stated in the Introduction the following: “Cyclophosphamide is an alkylating agent belonging to the group of oxazaphosporines. As cyclophosphamide is in clinical use for more than 40 years, there is a lot of experience using this drug for the treatment of cancer and as an immunosuppressive agent for the treatment of autoimmune and immune-mediated diseases. Besides antimitotic and anti-replicative effects, cyclophosphamide has immunosuppressive as well as immunomodulatory properties.”

The authors further elaborated: “Cyclophosphamide shows selectivity for T cells and is therefore now frequently used in tumor vaccination protocols and to control post-transplant allo-reactivity in haplo-identical unmanipulated bone marrow after transplantation. The schedule of administration is of special importance for the immunological effect: while cyclophosphamide can be used in high-dose therapy for the complete eradication of haematopoietic cells, lower doses of cyclophosphamide are relatively selective for T cells. Of special interest is the fact that a single administration of low-dose cyclophosphamide is able to selectively suppress regulatory T cells (Tregs). This effect can be used to counteract immunosuppression in cancer. However, cyclophosphamide can also increase the number of myeloid-derived suppressor cells. Combination of cyclophosphamide with other immunomodulatory agents could be a promising approach to treat different forms of advanced cancer.”

As stated from the Introduction above, the effect of a low dose CTX is different from that of a high dose of CTX. Therefore, the beneficial effects of FAS or HFS as presented here in this patent application against the harm caused by CTX cannot be predicted from the data from the published literature or Yen's prior work.

The authors emphasized that “Of special interest is the fact that a single administration of low-dose cyclophosphamide is able to selectively suppress regulatory T cells (Tregs).” This is of particular importance in view of the fact that Yen had previously reported that the administration of FAS can result in an increased concentration of WBC in the peripheral blood, including the lineages of lymphocytes, monocytes and granulocytes (Mao et al, Fibrinoplate-S for the Treatment of Radiation-induced Skin Damage; presented at the 61st Radiation Research Society Annual Meeting, Weston, FL, Sep. 19-23, 2015.)

However, the effects of FAS on regulatory T cells have never been studied. Therefore, before the disclosure here, it is not obvious that the administration of FAS may even have any effect on the concentration or the function of any T cells (including Treg cells) or other more specialized immune cells. It may even be possible that only certain doses of FAS or a particular regiment of injections may have any overall positive effects on the body. In addition, T cells are produced mainly in the thymus, which is a target of cyclophosphamide (see Feng et al, “Optimized Animal Model of cyclophosphamide-induced Bone Marrow Suppression”, in Basic & Clinical Pharmacology & Toxicology, 2016, 119; 428-435). The data presented here will show that the loss in weight of the thymus in mice treated with a high-dose CTX for 10 days can be mitigated by the administration of FAS 3 days before the administration of CTX. Further studies will be needed to evaluate the specific effect of FAS on subset of T-cells and whether the benefits of FAS are a direct effect on the residual cells in the thymus, or whether the beneficial effects are derived from bone marrow cells which migrate to the thymus to replace old or dying cells there, or the FAS may have other mechanism of actions which produce beneficial effects in addition to the direct medicinal effects on thymus cells.

The interaction of FAS-mediated mitigative effects with CTX, whether it is a high-dose CTX or low-dose CTX can be complicated and unpredictable, and certainly non-obvious. In one aspect, not only is the dose of FAS important, but also the timing of the FAS with respect to the timing of CTX administration. For example, in the same studies disclosed by Yen (Mao et al 2015) the beneficial effects are far greater if FAS is administrated at the time of harm (such as caused by irradiation) or after the harmful event. However, if FAS is administered before the harmful event, the medical benefits are much less obvious. This can be explained by the fact that certain harm e.g. irradiation, can damage stem cells—therefore, if stem cells were mobilized by FAS before the irradiation event, the irradiation will have a large number of stem cells to kill. In contrast, if the stem cells are activated after the irradiation event is gone, then all the mobilized stem cells will have a chance to go to the wound to regenerate into functional cells for the recovery of the wound.

However, the reverse is observed in the case of the mitigation by FAS against the harm caused by chemotherapy, even though chemotherapeutic drugs are also designed to kill fast-growing cells. A dose of FAS given before the administration of a well-known chemotherapeutic drug (specifically, CisPlatin) will result in much more benefit (i.e. less nephrotoxicity from the CisPlatin) than if FAS is given on the same day the CisPlatin is given (or 3 days after the CisPlatin dose): see patent application “Protein Nanospheres to treat Renal Failure, Dysfunction or Damage” submitted Jun. 3, 2024. This observation may be explained after the data is collected (and not before) by the hypothesis that CisPlatin (in contrast to irradiation) does not harm the rapid-growing stem cells—even though CisPlatin is designed to kill rapidly dividing cells such as cancer cells. According to this different hypothesis (different from that of the effect of irradiation on stem cells) if FAS is given 3 days before the administration of CisPlatin, the 3-day growth and mobilization of stem cells from the bone marrow will allow a large number of stem cells to reach the kidneys in time to repair any damage done by the CisPlatin that is administered on Day 0. Given the fact that in one case the benefit is best when FAS is given after the harm and in another case the benefit is seen only if FAS is given before the harm, it is obvious that the “hoped for” beneficial effects of FAS on the harm caused by CTX and how best (the timing) FAS should be administered against the harm caused by CTX are non-obvious.

The data on the best time to administer FAS against the harm caused by CisPlatin have been submitted to the USPTO. There are two U.S. provisional patent applications: The first application was received by the USPTO on Jul. 31, 2023, No. 63/628,513 with the title “Protein Nanospheres to Treat Renal Dysfunction.” The second provisional patent is titled “Protein Nanospheres to Treat Renal Dysfunction and Damage” which was submitted on Mar. 14, 2024. The U.S. non-provisional patent was titled “Protein nanospheres to treat renal failure, dysfunction or damage” submitted around Jun. 1, 2024.

In the present technology, the term “stem cell” or “progenitor cell” will include (a) “stem cells and related cells obtained from or residing in the bone marrow” (including CD34+ cells); (b) “primitive or progenitor cells or related cells obtained from or residing in tissue compartments outside of the bone marrow” such as cells on the endothelial walls of blood vessels in general, and in particular mesenchymal stromal cells or cells obtained from other organs or from any tissue. In other words, due to the different usages of these terms by different scientists, the term “stem cell” or “progenitor cell” in this disclosure would refer to “less mature” cells that will eventually differentiate into the fully mature and functional cells forming the tissues of an organ or a specific tissue, as found in healthy individuals.

It should be pointed out that the administration of FPS to animals has resulted in favorable oxidative/reductive changes in some cell populations even long after the intravenous administration of only one dose of FPS. Specifically, FAS has an effect on the oxidative reactivity of cells, either directly or indirectly. The production of reactive oxygen species (ROS) is reduced in spleen cells harvested from irradiated animals even on day 51 after one treatment with one dose of FAS (Mao, 2014). However, there was no discussion or expectation in that work to show that FPS may be beneficial to remedy the harm from chemotherapy or immunotherapy. In other words, the disclosure here relating the administration of FPS or HFS to mitigate the harm caused by a dual-agent (against cancer and against overactive immune system) is novel and non-obvious. It is within the realm of the present technology that FPS or HFS are expected to exert a positive effect on the repair of tissues damaged by dual-use agents by way of the beneficial effects of FPS as an inducer of anti-oxidants or its effect on the anti-oxidative pathway(s) inside cells.

Given the complexity of the actions of multi-valent agents on the host (in terms of treating the host's existing diseases) plus the inevitable side-effects caused by the multi-valent agent (harm by the multi-agent to the host) it is hard to expect that a single mitigation agent (such as FAS or HFS) can deal with both issues. In addition, the mitigating agent must not (a) decrease the effectiveness of the multi-agent to treat existing diseases; nor (b) worsen the disease entities (such as the proliferation of cancer cells or their metastasis, or the potency of the over-active immune cell system.) However, as this disclosure will reveal, the proposed product and the method disclosed here using protein nanospheres such as FAS and HFS to mitigate the harm caused by the multiagent can be both safe and effective.

Although details of the present technology including dimensions, concentrations and other exact measures of the present technology have been disclosed here, it should be recognized that people skilled in the art can use different and various other dimensions and parameters, which will still be infringements of the nature and spirit of the present technology.
1. It has been discovered according to the present technology that High-Fibrinogen Spheres (HFS) can be manufactured by coating blank albumin spheres with a solution containing human fibrinogen which is higher than 1.4 mg fibrinogen per mL of solution which also contains a high concentration of sodium tetradecyl sulphate of more than 5 mg per mL which is needed to keep the high concentration of fibrinogen molecules in a soluble state without precipitation in room temperature, the resulting HFS can be maintained for at least 1 year of stability in room temperature without flocculation. In the exemplary, room temperature can be appreciated as a general term for a comfortable range of temperatures in a living or working space, typically considered to be around 20-25° C. (68-77° F.).
2. It has been discovered according to the present technology that the intravenous infusion of nanoparticles such as FAS (fibrinogen coated albumin spheres made with fibrinogen solutions equal or less than 1.4 mg per mL in sodium tetradecyl sulphate of less than 5 mg per mL) or HFS into patients with one defined disease (including cancer or immune-related dysfunction) who have been treated with a multi-valent drug appropriate for the treatment of the single disease can result in the mitigation of the side-effects caused by the multi-valent drug.
3. It has been discovered according to the present technology that the intravenous infusion of nanoparticles such as FAS or HFS into patients with more-than-one defined diseases (including cancer and immune-related dysfunction) who have been treated with a multi-valent drug appropriate for the treatment of the more-than-one diseases can result in the mitigation of the side-effects caused by the multi-valent drug.
4. It has been discovered according to the present technology that the intravenous infusion of nanoparticles such as FAS or HFS into patients with one or more-than-one defined diseases (including but not limited to cancer and/or immune-related dysfunction) who have been treated with a multi-valent drug appropriate for the treatment of the disease(s) can result in the stimulation of stem cells from a variety of sites (including bone marrow and peripheral organs or tissues) which will result in the mitigation of the side-effects caused by the multi-valent drug.
5. It has been discovered according to the present technology that the intravenous infusion of nanoparticles such as FAS or HFS into patients with one or more-than-one defined diseases (including but not limited to cancer and/or immune-related dysfunction) who have been treated with a multi-valent drug appropriate for the treatment of the disease(s) can result in an improvement in the oxidative/reductive state of the patient which may result in the mitigation of the side-effects caused by the multi-valent drug.
6. It has been discovered according to the present technology that the intravenous infusion of nanoparticles such as FAS or HFS into patients with one or more-than-one defined diseases (including but not limited to cancer and/or immune-related dysfunction) who will be treated with a multi-valent drug appropriate for the treatment of the disease(s) can obtain the best mitigative effects against the harm caused by the multi-valent agent when the nanoparticles are administered before the onset of the treatment with the multi-valent agent.
7. It has been discovered according to the present technology that the intravenous infusion of nanoparticles such as FAS or HFS into patients with one or more-than-one defined diseases (including but not limited to cancer and/or immune-related dysfunction) who will be treated with certain multi-valent drugs appropriate for the treatment of the disease(s) can obtain the best mitigative effects against the harm caused by the multi-valent agents when the nanoparticles are administered after the onset of the treatment with the multi-valent agent.
8. It has been discovered according to the present technology that certain blood-organ barriers, including blood-brain barrier and blood-thymus barrier which can be harmed by chemotherapy or irradiation or any toxic substances can be mitigated by the administration of FAS or HFS.
9. It has been discovered according to the present technology that the dose of FAS or HFS that can be safely administered intravenously to patients who can benefit from such administrations can exceed 160 mg per kilogram, including up to 320 mg per kilogram weight of the patient.

### EXPERIMENTS

### Experiment One: Manufacture of Protein Nanoparticles with High Fibrinogen Concentrations, i.e. High Fibrinogen Spheres (HFS)

### INTRODUCTION

The Applicant has disclosed extensively the manufacture of fibrinogen-coated albumin spheres (FAS) suspended in a physiologically compatible solution, to result in a ready-to-use biological product to treat patients; the biological product being called Fibrinoplate-S(FPS). See Patent number 6264988 “Fibrinogen-coated Microspheres” filed on Jun. 4, 1998; and issued on Jul. 24, 2001, inventor is Yen. The spheres suspended in this FAS suspension have an AVERAGE fibrinogen content of 5% w/w, i.e. on the average, 5 mg of fibrinogen molecules are attached to 100 mg of the fibrinogen-coated albumin spheres. We intend to manufacture by a novel method here a new preparation called HIGH FIBRINOGEN SPHERES, which will have on average a fibrinogen content of higher than 5%, e.g. 10% or 10 mg fibrinogen per 100 mg of HFS spheres.

It is generally known that fibrinogen is a cryo-protein in that it is very sensitive to the effect of temperature. Fibrinogen is made in the body at body temperature which is 37 deg C. Before isolation from the plasma to be used for biological assays or being manufactured into other products—up until then, the fibrinogen molecules have never been reduced to room temperature. In room temperature or colder temperature, a solution containing a high concentration of fibrinogen can easily clump, often irreversibly. Therefore, fibrinogen products sold in the market typically contain a high concentration of salt (a) first to make sure the fibrinogen molecules remain in a soluble state before lyophilization; (b) when a dry-freeze powder of fibrinogen is to be reconstituted, even if the consumer adds back only water, the excipient of the powder already has enough salt to keep the fibrinogen molecules in the dissolved state. For example, Sigma sells a commercial product called “Fibrinogen from human plasma” (product F3897). The label states that it contains about 15% sodium citrate and about 25% of sodium chloride. That means in one gram of the powder, only 0.6 gram is protein of which only about 80% is clottable protein (fibrinogen.) It has been found in these experiments that this high concentration of salt will aggregate the suspension of blank spheres when the fibrinogen solution (containing either sodium citrate or sodium chloride, or both) is added to the suspension of blank spheres to coat the spheres with fibrinogen. Therefore, such preparations are a failure and cannot be used for any experiments, far less for the treatment of patients.

However, it is found here that when the fibrinogen molecules are dissolved in a solution containing sodium tetradecyl sulphate (STS), the fibrinogen molecules can stay in solution even at concentrations as high as 50 mg per mL. Furthermore, the fibrinogen solution can be frozen; and when it was warmed back to 37 deg C., the fibrinogen molecules remained in solution.

Yen has disclosed that surfactants can have two effects: (a) the presence of surfactants during the manufacture of the blank albumin spheres can have a profound effect on the size of the blank spheres (whether these blank spheres will be coated with fibrinogen or not, or with other biological molecules); the presence of surfactants will increase the size of the spheres compared to when surfactants are absent during the production of blank spheres; (b) the effect on the solubility of fibrinogen in high fibrinogen concentrations is a different phenomenon from the effect of surfactant in the size of the spheres. In particular, STS is effective in keeping fibrinogen molecules in solution. Here, the detergent STS is used mainly to maintain the fibrinogen molecules in a soluble state in room temperature and is not used in the steps to create the blank spheres. In fact, addition of detergents or surfactants is not needed in the manufacture of the blank sphere suspension, particularly spheres smaller than one micron in diameter.

Small-sized spheres (less than one micron in diameter) have many advantages compared to large spheres (defined as any sphere larger than 5 microns in diameter.) When small spheres are desired, addition of any surfactant to the albumin solution during the steps of making the blank spheres can be counterproductive. This is because the presence of detergents or surfactants in a human albumin solution during the step of making the blank spheres will increase the size of the resulting spheres, which is not desirable in the present technology. Many of the medical benefits of FAS lie with the fact that the fibrinogen molecules on the surface of the FAS interact with and will bind to specific cells in the body for their medicinal effects. Increasing the size of the spheres (coated or not) will (a) decrease the number of spheres per mg of albumin used because a large sphere has a larger mass; (b) rheological principles will dictate that larger particles will move along the blood stream close to the center of the flow, while smaller particles move closer to the wall—and it is the endothelial wall where wounds occur; thus allowing the FAS to react closer to the site of injury when FAS is to be used to control bleeding. The small size of the average FAS sphere (less than 200 nanometer) may be another factor why these nanoparticles can penetrate the barriers in the bone marrow to reach the stem cell compartment to mobilize the stem cells from there to move into the blood compartment.

The economy of producing smaller spheres is obvious because the same mass of starting material (in terms of the mass of albumin as a starting material) can result in more small particles than large particles; and the small particles have more surface area than the same mass of large particles. This is obvious because the formula for surface area is “4πR²” but the formula for the mass of a sphere is “4/3πR³”. Therefore, in all of recent disclosed methods by Yen, the step of making blank spheres do not involved any surfactants, but the solution containing fibrinogen typically contains the detergent STS at a concentration of 1 mg per mL of solution before the solution is added to the suspension of blank spheres, to product FAS. We intend to keep the spheres small (below 200 nanometer) while increasing the fibrinogen content (fibrinogen concentration per sphere.)

### Purpose:

To produce High-Fibrinogen Spheres (HFS) which contain a higher concentration of fibrinogen per sphere as compared to FAS (fibrinogen-coated albumin spheres)

For the discussion here and elsewhere in this disclosure, FAS refers to the fibrinogen-coated albumin spheres made by using fibrinogen solutions at 1.4 mg fibrinogen dissolved in STS1 (which means STS at a concentration of 1 mg per mL dissolved in water) to coat the blank spheres. HFS means the High-Fibrinogen Spheres made in the method disclosed here. The steps in making the blank spheres are the same as disclosed in Yen, patent U.S. Pat. No. 6,264,988. FAS typically contains on an average about 5% w/w, i.e. 5 mg of fibrinogen per 100 mg of FAS spheres.

It has been noted that in previous batches of FAS: when the FAS was made with fibrinogen concentrations higher than 1.4 mg per ml, dissolved in an STS solution less than 1 mg per ml, the spheres may flocculate in the bottle after storage at room temperature for more than one month. Therefore, this experiment will evaluate the benefit of having all fibrinogen solutions dissolved at STS1.5 at the time the fibrinogen solution was to be mixed with the suspension of blank spheres.

### Materials and Methods

Production of medical-grade human fibrinogen: units of medical-grade “cryo-precipitate” were purchased from a certified vendor which sells these units to hospitals for infusion readily into patients who need them. The units were processed into a high concentration of fibrinogen using a proprietary method invented by Dr. Yen with a purity of greater than 95%, which means more than 95% of the protein in the solution was clottable fibrinogen.

The freshly purified fibrinogen solution was adjusted to the following concentrations in either STS1.5 (meaning a concentration of 1.5 mg STS dissolved per mL of water) or STS7.5 (at a concentration of 7.5 mg STS per mL.) Table 7 lists the various fibrinogen preparations which may be further processed before they are used to coat the blank spheres.

TABLE 7
Preparations of Fibrinogen (Fbg) to be used to make HFS or FAS

| | | SOLVENT | INITIAL | |
|---|---|---|---|---|
| | | USED TO | CONC | |
| | | ADJUST | OF FBG, | |
| | | FBG | mg per | |
| PREPARATION | PURPOSE | SOLUTION | mL | STEPS |
| A | Evaluate the | STS7.5 | 50 | To be frozen for 1 month |
| | effect of freezing | | | and then thawed for use |
| | | | | in lower concentrations |
| | | | | of Fbg and STS |
| B | Evaluate the | STS7.5 | 7 | Keep in 37 deg C. for 24 |
| | effect of warming | | | hr, then diluted to |
| | to 37 deg C. in | | | STS1.5 and 1.4 mg Fbg |
| | high STS conc | | | per mL, with water at |
| | | | | room temperature |
| C | Evaluate the | STS7.5 | 14 | Keep in 37 deg C. for 24 |
| | effect of warming | | | hr, then diluted to |
| | to 37 deg C. on a | | | STS1.5 and 2.8 mg Fbg |
| | high Fbg and | | | per mL, with water at |
| | high STS | | | room temperature |
| | solution | | | |
| D | Effect on making | STS1.5 | 1.4 | STS is higher than |
| | useful FAS using | | | Standard solution as |
| | solution kept in | | | disclosed by Yen |
| | room temp | | | previously |
| E | Effect on making | STS1.5 | 2.8 | STS is higher and Fbg is |
| | useful HFS by | | | 2x that of standard Fbg |
| | using solution | | | conc as disclosed by Yen |
| | kept in room | | | previously |
| | temp | | | |

The purpose of Preparation A is to evaluate the effect of freezing on a high concentration of Fbg (at 50 mg per ml) dissolved in a high concentration of STS7.5 (defined as STS at 7.5 mg dissolved in water.) After frozen at −20 deg C. (freezer) for over one month, the content inside the tube containing the frozen Fbg solution was thawed directly in a 37 deg C. water bath. Then 10 mL of the thawed solution was added 40 mL of water to adjust the concentrations to: STS to 1.5 mg per mL and Fbg at 10 mg per mL. To keep the STS concentration steady at 1.5 mg per mL, further dilutions were done with STS1.5—to create two solutions: one equivalent to Preparation D (with Fibrinogen concentration at 1.4 mg per mL, called A-d, to make FAS) and another equivalent to Preparation E (with Fibrinogen concentration at 2.8 mg per mL, called A-e, to make HFS).

Preparation B and C were made to evaluate the effect of heating the solutions to 37 deg C. for at least 24 hours. Then they were diluted with 4× volume of water to create two solutions: respectively containing 1.4 and 2.8 mg Fbg per ml (both now at concentrations of STS at 1.5 mg per mL).

Preparation D and E were kept at room temperature until used to coated blank spheres.

The volume of all the fibrinogen solutions (Preparation A, B, C, D, E after adjustment to the desired Fbg concentration and the desired STS concentration) to be used is 1 vol of the Fbg solution added quickly at room temperature per 3 vol of blank spheres in room temperature.

The yields of the reactions were measured by (a) centrifugation of the spheres from the suspensions to remove the spheres in order to obtain the non-turbid Supernatant Fraction (SF); (b) assay of the protein concentrations in the Whole Suspension (WS) versus the Supernatant Fraction (SF). The concentration of Spheres is obtained from the formula: Protein concentration in the WS minus the protein concentration in the SF. (c) the yield of the batch is the “Protein concentration of the Spheres divided by the Protein centration in the WS.”

### Results

Suspensions of blank spheres were produced successfully by using excipient grade of human serum albumin purchased from a licensed vendor. The suspension contained 11.2 mg of blank spheres per ml of suspension, before the addition of each of the adjusted fibrinogen solutions B, C, D, and E. The Fbg solutions (room temperature) were mixed quickly with the suspension of blank spheres (room temperature) at a ratio of 1 vol of the Fbg solution per 3 vol of blank spheres. The yield of all the preparations was similar: at about 82%. Examination under the microscope showed no signs of aggregation or the presence of spheres larger than 5 microns. Therefore, all the adjusted preparations (B, C, D, E) were acceptable for manufacture of FAS and HFS.

Regarding the effect of freezing on Preparation A. After a month or longer of being kept in the frozen state, the solution of high Fbg (50 mg per mL in STS7.5) had no problem re-dissolving. After dilution with water to reduce the STS7.5 to STS1.5; and further dilution with STS1.5 to create Preparation A-d (similar in composition to Prep D) and Preparation A-e (similar in composition to Prep E) the Fbg solutions were used to coat a new batch of blank spheres. The yield of the FAS or HGS from Prep A-d and A-e were similar to those from Prep D and Prep E, respectively. There were no aggregates or spheres larger than 5 microns in diameter in these preparations.

However, if Preparation A was diluted with Normal Saline (NS) to become a Fbg solution (1.4 mg per mL of NS) the addition of this preparation of Fbg with blank spheres will cause immediate aggregation of spheres which will render the suspension useless or even harmful.

### Comments

The data showed that Fibrinogen solutions at as high a concentration as 50 mg Fbg per mL of STS7.5 can be frozen, thawed and used to make FAS as well as HFS with no detectable problem. The FAS and HFS will be used in the following experiments to assess their medical effects in the mitigation of the harm caused by a multi-agent such as CTX.

Although only two concentrations of Fbg were used here as coating solutions, i.e. 1.4 and 2.8 mg per mL (both in STS1.5) we expect that concentrations of Fbg higher than 2.8 mg per ml can be used to make HFS that may be useful in this or other medical conditions, while coating solutions of Fbg lower than 1.4 mg per ml may be useful in other applications. The effect of STS in higher or lower concentrations than 1.5 mg per mL in the coating solution may also have an effect on the binding of Fbg onto the spheres. The effect cannot be predicted before specific experiments are performed for all the various different kinds of disease conditions.

For the convenience of discussion, we will state the FAS fibrinogen content is 5% w/w; and the HFS fibrinogen content is 10% w/w. But these are only average values. Each individual sphere within the suspension may have different fibrinogen contents, although the fibrinogen content is expected to be within a narrow range.

### Experiment Two: Mitigation by Protein Nanoparticles of the Harm Caused by Cyclophosphamide in Healthy Animals

### Introduction

Nanoparticles such as FAS has been known to accelerate the healing of wounds caused by irradiation or in bone fractures. The experiments below aim at finding out the effectiveness of FAS and HFS on the mitigation of the harm caused by CTX to various organs in healthy (no cancer nor immune disease) animals.

### Materials and Methods

We followed the model published by Feng et al in Basic and Clinical Pharm & Tox, 2016:119, 428-435; “Optimized Animal Model of CTX-induced Bone Marrow suppression.”

A high dose of CTX was used here. There were 4 treatment groups: all the mice were sacrificed on day 12 and day 17. Number of mice: 10 per group: 5 sacrificed on day 12, 5 sacrificed on day 17. The 4 groups were:
1. Saline Control Group (no FPS, no CTX): IP 2 ml per kg per day, ×10 days
2. Mitigation Group: CTX treatment (50 mg/kg, daily IP ×10 days; same as group 3,4) FPS was given on Day 1 about 2 hours after the start of CTX treatment.
3. Prophylaxis Group: FPS plus CTX (FPS at 16 mg per kg, given 1 dose on day −3) CTX treatment same as group 2.
4. CTX group: no FPS

Data to be collected included:
a. Weight of whole animals q day
b. Weight of thymus (sectioned for staining only if there is significant difference between Group b and Group c)
c. Blood counts: RBC, WBC, platelets on day 12 and day 17.
d. Cell staining on the thymus biopsy: including CD34+ cells and T-effector cells, T-helper cells, T-regulator cells.

### Results

The experiment is conducted with FAS, to be repeated by using HFS instead of FAS after cell staining are completed.

The following are the results of using FAS for mitigation of the cellular and organ damage by a high dose of CTX.

### Pilot Study—the Effect of Fps on the Toxicity of Cyclophosphamide in Male Cd-1 Mice

### Results on Survival

Three animals died in group 2 (FBS 2 hours after 1st CTX) on days 9, 10 and 12, respectively.

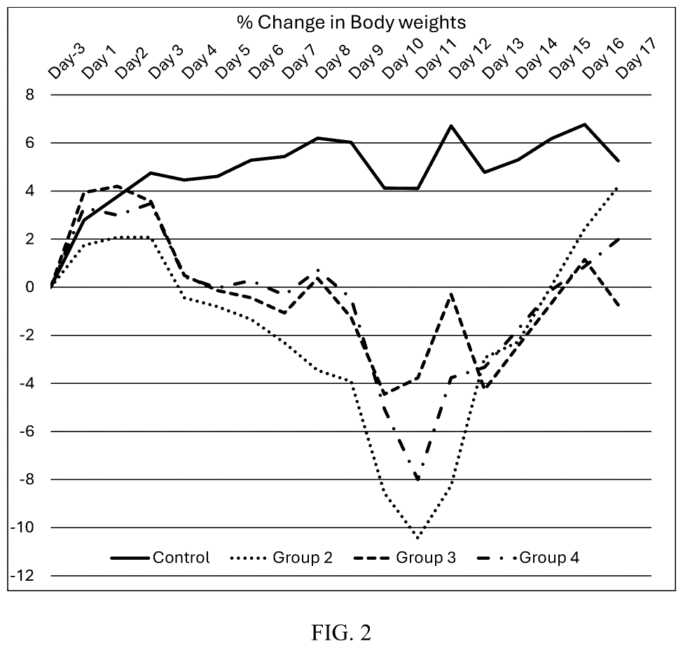

Referring to FIG. 2, the CTX dosing induced a profound effect (confirming that the model worked very well) as the saline dose group (Group 1) body weight increased compared to the background (Day −3) while all CTX treatment group showed decrease in the body weights after Day 1 which is the starting date of CTX treatment. There was no evident improvement in body weights of FPS-treated groups (Group 2 and 3) compared to Group 4 (no FPS). On the first 2 days after CTX termination (i.e. days 11 and 12) the changes in the body weights seemed to recover better in group 3, However, this recovery was not statistically significant given the small number of animals per group.

### Results in Thymus and Spleen Weights on Day 12 and Day 17

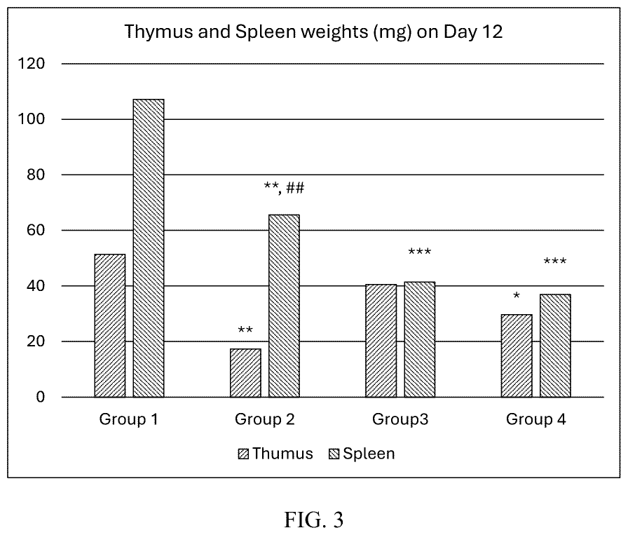

Referring to FIG. 3, where * is P<0.05, ** is P<0.01, *** is P<0.001 compared to the control group, and ## is P<0.01 compared to the Group 4.

The data showed that Group 3 has thymus weight comparable to that of Control Group 1. All groups except Group 3 had statistically significantly lower thymus weight compared to Group 1. The difference between thymus weight in Group 3 and 4 are significantly different.

All spleens were statistically significantly lower than control but the Mean spleen weight in Group 2 was statistically significantly higher than in group 4 (when included dead animals).

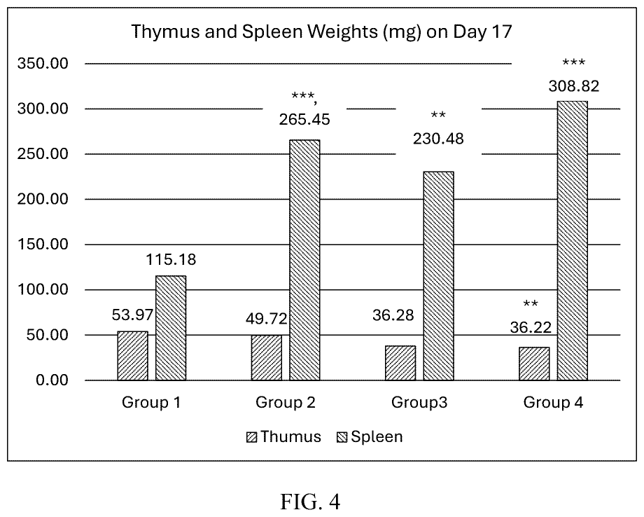

FIG. 4 shows the thymus and spleen weights in the 4 groups. The spleen weighs in all dose groups became higher approximately 2-3 fold) than in the control group (probably due to overcompensation recovery). The closest to control levels were seen in group 3. The thymus weights were still lower in all CTX dosed groups but was statistically significantly lower only in group 4 (not FPS treatment).

Referring to FIG. 4, where ** is P<0.01, and *** is P<0.001 compared to the control group.

Since we expect Prophylasis (Group 3) to be better than Mitigation (Group 2) in terms of the mitigation of the harm caused by CTX (Group 4), we will focus on the differences between Group 3 and Group 4.

Regarding Thymus weight: although the mean value of the thymus weight in Group 3 (36.28 gram) is very similar to that of Group 4 (36.22), however, due to the difference in the variation within the groups, the Group 3 value is NOT significantly different from Group 1, but significantly different from Group 4.

Regarding Spleen weight: the heavier the spleen, the greater degree of splenomegaly or compensatory response to injury either in the spleen or in other organs (e.g. Thymus). The data show that while Group 3 spleen weight is higher than that of Group 1, it is in between that of Group 1 and Group 4. The P value showed that the spleen weight is significantly different between Group 3 and Group 4.

The overall result in the weight of thymus or of spleen show that FPS given 3 days before the start of CTX in mice has a definite mitigative effect.

The organ weights (of thymus or of spleen) are overall parameters or indices (and not specific) of the beneficial effects on FPS in animals treated with CTX. We will study by specific cell-staining (in the bone marrow, in spleen, in thymus and other tissues) on what cell types are injured by CTX and how a dose of FAS given 3 days before CTX treatment can cause better and faster recovery of these CTX-targeted cells.

### Results on Hematology Results

PLT were not statistically changed in any dose group either on Day 12 or 17.

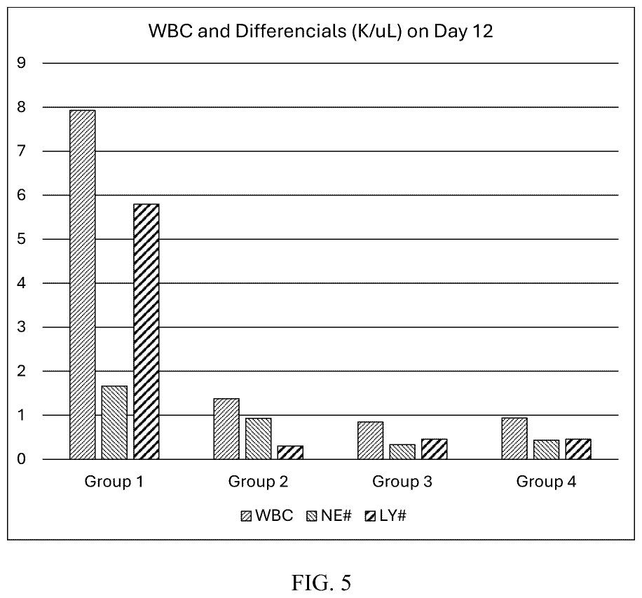

FIG. 5 shows the concentration of White Blood Cell (WBC) and their subgroups (“differentials”). For the comparison of the various groups: See statistics in the numerical Table 8 below where ** is P<0.01, and *** is P<0.001 compared to the control group.

TABLE 8
White Blood Cell and their subgroup concentrations on Day 12:

| | Group 1 | Group 2 | Group 3 | Group 4 |
|---|---|---|---|---|
| WBC | 7.93 | 1.37*** | 0.844*** | 0.936*** |
| NEUTROPH# | 1.66 | 0.93 | 0.33*** | 0.43*** |
| LYMPHOCYTE# | 5.80 | 0.3** | 0.456** | 0.456*** |
| PLT | 990.20 | 672.33 | 813.60 | 887.80 |

The data show that all the CTX groups (Group 2, 3, 4) had WBC significantly lower than that of Group 1. This is evidence that CTX has myelosuppressive effects. Data from bone marrow staining is still pending. But WBC are mainly produced in the bone marrow: therefore peripheral blood count reflects the injury in the bone marrow. The data is consistent with CTX toxicity in the bone marrow, although the harm apparently is negligible on the lineage leading to Platelet production, because platelet counts in the peripheral blood in this experiment is not affected by this dosing regiment of CTX.

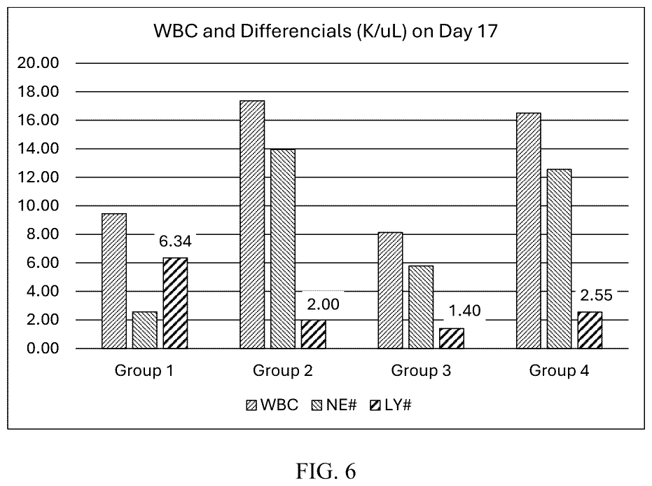

FIG. 6 shows the White Blood Cell (WBC) count including the differentials on Day 17. For comparison of the various groups, see statistics in the numerical Table 9 below, where * is P<0.05, ** is P<0.01, and *** is P<0.001 compared to the control.

TABLE 9
White Blood Cell counts including the subgroups

| | Group 1 | Group 2 | Group 3 | Group 4 |
|---|---|---|---|---|
| WBC | 9.44 | 17.37 | 8.13 | 16.51 |
| NE# | 2.56 | 13.94* | 5.78 | 12.55 |
| LY# | 6.34 | 2** | 1.39*** | 2.55*** |
| % Lymph | 69.25 | 10.93 | 18.18 | 18.41 |
| PLT | 1033.00 | 877.50 | 801.40 | 886.67 |

A comparison of Table 8 and Table 9 will show that there is rapid recovery of peripheral blood cells regarding WBC between day 12 and day 17. Whereas the peripheral WBC on Day 12 is only 0.844 and 0.936 for Group 3 and 4, respectively, the WBC have recovered to supra-normal values of 8.13 and 16.51, respectively. The fact that WBC on Day 17 in Group 4 greatly overshot its recovery (rising from 0.936 to 16.51) within 5 days may be a reflection of the greater harm done to the WBC lineage in the bone marrow by CTX in the absence of FAS (i.e. Group 4 has no FPS, while Group 3 has FPS to mitigate the harm of CTX on the bone marrow.)

Also, regarding NE concentration, the recovery of neutrophils by Day 17 shows that the concentration of these cells in Group 3 (5.78) is between that of the control value (group 1, 2.56) and that of the CTX-only Group 4 (12.55). The data is consistent with the mitigative effect of FPS on CTX toxicity on the lineage producing neutrophils.

### Results on the Effect on Red Blood Cells (RBC)

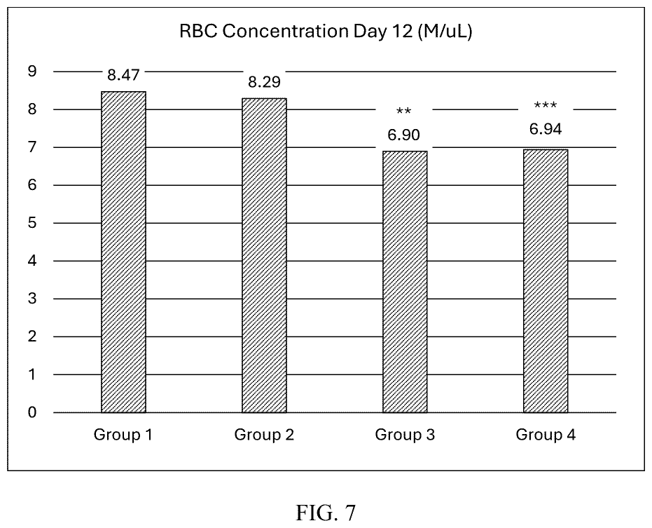

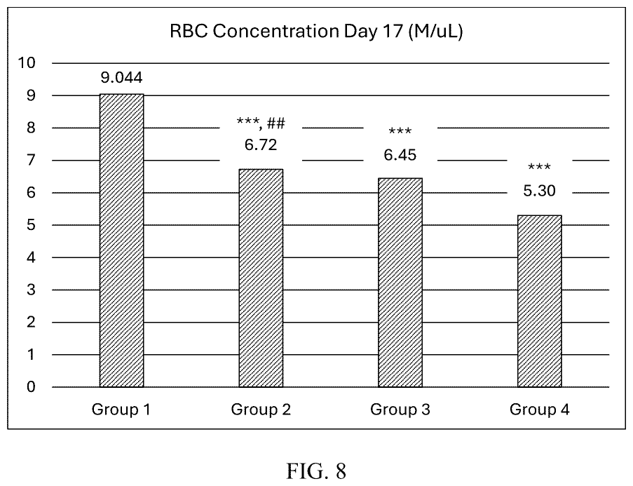

FIGS. 7 and 8 shows the concentration of RBC on Day 12 and Day 17, respectively, where * is P<0.05, ** is P<0.01, *** is P<0.001 compared to the control group, and ## is P<0.01 compared to the Group 4.

The data show that the concentration of RBC in the peripheral blood did not fully recover from the toxic effect of CTX even by Day 17. Again, on Day 17, the concentration of RBC in Group 3 (6.45) is valued in between that of the control (Group 1, 9.044) and Group 4 (5.30)

The hematology parameters did not return to normal by Day 17 and may continue to drop after Day 17. Collection of data may need to go beyond (longer than) Day 17 to show the different effects of CTX on different cells; and the differential mitigative effect of FAS on the harm caused by CTX.

We are mindful that different cell types will play different roles in the health of the patient; and they may have varying effects on patients with cancer or with autoimmune problems. The effects of any harmful agent on these cells are further complicated by the dose-effects of CTX (as shown by various scientists that low-dose CTX has different effects than high-dose CTX.) Therefore, we expect various doses of FAS can have various effects on the mitigation of CTX toxicity, as well as the independent effects of FAS on various cell types towards the recovery or normalization of health of the affected patients. We expect that HFS can have even more pronounced positive effects on the various stages of healing on cancer patients and autoimmune patients, when compared to the effects of FAS.

The data here confirmed the importance of the timing of the administration of FAS with respect to the first dose of CTX to be given to the patient. Even though we used the same protocol here as in previous studies in the mitigation of FAS on the toxicity of CisPlatin (i.e. FAS given 3 days before the start of the chemotherapy is beneficial) we expect that the optimal dosing time may be different in each case (e.g. can be better when given earlier than day −3) due to the different mechanisms of action between CTX and CisPlatin. Further studies are needed to show the best dose and the best timing of the administration of FAS or HFS for the mitigation of various types of chemotherapy.

### Further Comments

The thymus is a very complicated organ. The anatomy of this organ was well described in a chapter called “Thymus histology” (www.kenhub.com/en/library/anatomy/histology-of-the-thymus) authored by Crumble (Jul. 21, 2023).

The author explained: “Production of immune cells primarily occurs in the bone marrow; however, education of a special lineage of immune cells takes place in the thymus.”

The following Table 10 was taken from the chapter, which outlined the various cells in the different compartments of the thymus. Of particular interest is the fact that the thymus maintains a “blood-thymus barrier” so that antigens in the blood do not easily get into the thymus to disturb the normal education of naive cells there.

TABLE 10
Key facts about the histology and function of the thymus
Key facts about the histology of the thymus

| | |
|---|---|
| Structure | Divided into thymic lobules separated by connective tissue septae. Each |
| | lobule is made up of a peripheral cortex and an inner medulla. |
| Thymic cortex | Superficial layer: superficial subscapular cells forming a squamous sheath |
| | and a blood thymus barrier |
| | Middle layer: stellate thymic epithelial and cytoreticular cells |
| | Inner layer: squamous cortical thymic epithelial cells which form the |
| | corticomedullary barrier |
| Thymic medulla | A second layer of squamous thymic epithelial cells and cytoreticulum |
| | Thymic epithelial cells congregated into Hassall's corpuscles |
| | Thymic nurse cells which are responsible for educating thymocytes |
| Function | Maturation and education of T lymphocytes via positive and negative |
| | selection |

The data disclosed here did not discuss the effect of CTX on various cells. The study will be continued to see what cells types are affected by CTX and are protected by FAS administration. To be more specific, we intend to study: (a) the toxic effect of CTX on the various cells and different structures in the thymus, and (b) which cell type and what location these “CTX-targets” exist, which would benefit from FAS and HFS mitigation. However, the data showed that the gross morphology and weight of the FAS-treated groups (i.e. Group 2 and 3) are improved compared to the CTX-only group. We believe that follow-up studies on the histology of the thymus from the 4 groups will provide data on the specificity of FAS and HFS mitigation against the harm done by CTX.

We also intend to conduct experiments showing that FAS and HFS can mitigate the harm done to the “blood-thymus barrier.” This approach has been described in J Exp Med. 1972 Sep. 1; 136(3):466-498. doi: 10.1084/jem.136.3.466. The title is “Evidence for a Blood-Thymus Barrier Using Electron-opaque Traces” authored by Raviola and Karnosky. The tracers mentioned in that article included horseradish peroxidase, cytochrome c, catalase, ferritin, colloidal lanthanum. We expect that CTX will harm the blood-thymus to some degree but such harm can be mitigated by the administration of FAS or HFS.

This disclosure focuses on the mitigation of the harm done by agents to the blood-thymus barrier. We fully expect that any agent that can harm any blood-organ barrier, including blood-brain barrier can be mitigated by the administration of FAS or HFS. The harmful agents are not limited to CTX which is only an example of a harmful agent. The agents can be chemotherapy, irradiation, immune-suppressive drugs or any toxic substance that can harm such blood-organ barrier.

### Follow-Up Experiments

Given the complexity of the disease states and the challenge in understanding the molecular effect of the treatment agents, it is expected that a large number of experiments need to be conducted to elucidate how FAS or HFS can mitigate the harmful effect of the treatment agent without (a) decreasing the effectiveness of the treatment agent on the disease states, and (b) without increasing the severity of the disease conditions. The following Table 11 is a logical presentation of the approach to be taken for further study of the beneficial effects of FAS and HFS.

TABLE 11
Overall view of a systematic approach to evaluate the mitigative effect of nanoparticles
against the harm on a specific organ from a multi-valent treatment agent such as CTX

| Categories | Disease States | Treatments | Expected Results |
|---|---|---|---|
| A1 | No Disease, | CTX ALONE | Thymus weight and histology; and cells |
| | Normal subjects | | in peripheral blood and bone marrow |
| | | | show damage compared to saline control |
| A2 | | CTX + FAS | Thymus weight and histology; and cells |
| | | | in peripheral blood and bone marrow |
| | | | show less damage than A1 |
| A3 | | CTX + HFS | Thymus weight and histology; and cells |
| | | | in peripheral blood and bone marrow |
| | | | show less damage than A1 |
| B1 | Autoimmune | CTX ALONE | Immune status improved, but thymus |
| | patients or patients | | weight and histology; and cells in |
| | with immune | | peripheral blood and bone marrow show |
| | dysfunctions, no | | damage compared to saline control |
| B2 | cancer | CTX + FAS | Immune status improved and thymus |
| | | | weight and histology; and cells in |
| | | | peripheral blood and bone marrow show |
| | | | less damage than B1 |
| B3 | | CTX + HFS | Immune status improved and thymus |
| | | | weight and histology; and cells in |
| | | | peripheral blood and bone marrow show |
| | | | less damage than B1 |
| C1 | Cancer patients, | CTX ALONE | Cancer status improved, but thymus |
| | without immune | | weight and histology; and cells in |
| | problems | | peripheral blood and bone marrow show |
| | | | damage compared to saline control |
| C2 | | CTX + FAS | Cancer status improved; thymus weight |
| | | | and histology, and cells in peripheral |
| | | | blood and bone marrow show less |
| | | | damage than C1 |
| C3 | | CTX + HFS | Immune status improved; thymus weight |
| | | | and histology, and cells in peripheral |
| | | | blood and bone marrow show less |
| | | | damage than C1 |
| D1 | Immune-suppressed | CTX ALONE | Cancer and immune status improved, but |
| | or compromised | | thymus weight and histology; and cells |
| | patients who also | | in peripheral blood and bone marrow |
| | have cancer | | show damage compared to saline control |
| D2 | | CTX + FAS | Cancer and immune status improved; |
| | | | thymus weight and histology, and cells in |
| | | | peripheral blood and bone marrow show |
| | | | less damage than D1 |
| D3 | | CTX + HFS | Cancer and Immune status improved; |
| | | | thymus weight and histology, and cells in |
| | | | peripheral blood and bone marrow show |
| | | | less damage than D1 |

In this disclosure we use CTX as the example of a treatment agent. However, it is readily understood that any other chemotherapy, irradiation, drug or toxic substance can be the treatment agent for diseases, which harm can be mitigated by the present technology. Also, we did not identify the specific dose of CTX to be evaluated. Different doses (high versus low) of the treatment agent must be evaluated if existing data show a non-linear (or unexpected) response of the body to the dosage of the treatment agent (i.e. a high dose does not bring about a high positive response, even within the “safety margin” of the agent.) This is particularly important if a low dose brings about a positive response while a high dose of the treatment agent brings about a negative response.

We also used the thymus as an indicator organ regarding the mitigative effect of FAS or HFS against the harm done to this organ by muti-agents designed to treat diseases. The mitigative effect of FAS or HFS against the harm of a treatment agent on other organs should be studied, if existing data already show that such organs will be harmed in the absence of FAS or HFS.

In Table 11, we listed four main categories (A, B, C, D) with 3 subcategories (e.g. A1, A2, A3). We recognize that there should be a fourth, fifth and sixth sub-category (e.g. A4, A5, A6; not listed) which is FAS alone (without CTX), HFS alone (without CTX), and saline control, respectively. However, we expect no harm on the patient from FAS and HFS alone: they should have the same effect from that of a dose of normal saline on the patients (same volume administered as FAS or HFS.)

The present technology discloses the manufacture of fibrinogen-coated albumin spheres (FAS) and High-Fibrinogen Spheres (HFS) which have higher concentrations of fibrinogen molecules per sphere than FAS, and their use for medical treatments. Both kinds of nanoparticles are effective in the mitigation of the toxic effects of certain chemotherapeutic and radiological agents that are typically used in the treatment of cancer, or the treatment of autoimmune diseases, or for patients with both diseases. FAS and HFS can exert their beneficial effects via a variety of mechanisms which match the need of the body for specific cell types, including any of the subgroups of T cells and antibody producing cells, the relative concentration of each kind is vital to the balance between tumor surveillance and autoimmune disease suppression.

According to one aspect, the present technology can include a method of using an albumin nanoparticle suspension containing submicron albumin spheres to mitigate damage to tissue by administration of a multi-valent agent to a subject in need thereof. The method can include the step of administering intravenously a therapeutically effective amount of the albumin nanoparticle suspension containing the submicron albumin spheres to the subject. The albumin spheres can be configured to augment a function or effectiveness of stem cells or precursor cells in vivo to mitigate damage to the tissue caused by treatment to the subject by the multi-valent agent.

According to another aspect, the present technology can include a method of treating one or more side-effects from administration of a multi-valent agent to a patient in need thereof with albumin nanoparticles. The method can include the steps of providing a suspension including fibrinogen-coated albumin nanospheres prepared by coating blank albumin spheres with a solution containing human fibrinogen that is greater than 1.4 mg fibrinogen per mL of the solution. Administering intravenously the suspension to the patient at a concentration of the fibrinogen-coated albumin nanospheres sufficient to augment a function or effectiveness of stem cells or precursor cells in vivo to at least mitigate damage caused by administration of the multi-valent agent to the patient.

According to yet another aspect, the present technology can include a suspension for mitigating damage to tissue caused by administration of a multi-valent agent to a patient. The suspension can include fibrinogen-coated albumin nanospheres prepared by coating blank albumin spheres with a solution containing human fibrinogen that is greater than 1.4 mg fibrinogen per mL of the solution.

In some embodiments, the multi-valent agent can have any one of or any combination of a chemotherapeutic effect and an immunosuppressive effect to the subject or patient.

In some embodiments, the albumin spheres can be configured to have an effect on an oxidative reactivity of the stem cells or precursor cells.

In some embodiments, the albumin spheres can be configured to have an effect on the stem cells or precursor cells as an inducer of anti-oxidants or on an anti-oxidative pathway inside the stem cells or precursor cells.

In some embodiments, the albumin spheres can be configured to maintain a mass of a thymus of the subject or patient, and cells in the thymus.

In some embodiments, the administering of the albumin nanoparticle suspension can be prior to an onset of treatment of the multi-valent drug to the subject or patient.

In some embodiments, the administering of the albumin nanoparticle suspension can be after an onset of treatment of the multi-valent drug to the subject or patient.

In some embodiments, the albumin spheres of the albumin nanoparticle suspension can be bound with fibrinogen molecules to produce Fibrinogen Albumin Spheres (FAS).

In some embodiments, the fibrinogen albumin spheres can be High-Fibrinogen Spheres (HFS) prepared by coating blank albumin spheres with a solution containing human fibrinogen that is greater than 1.4 mg fibrinogen per mL of the solution.

In some embodiments, the solution can contain a concentration of sodium tetradecyl sulphate greater than 5 mg per mL of the solution.

In some embodiments, the sodium tetradecyl sulphate can be configured to keep fibrinogen molecules in a soluble state without precipitation at room temperature.

In some embodiments, the administering of the albumin nanoparticle suspension to the subject or patient can be at a dose greater than 160 mg per kilogram weight of the subject or patient.

In some embodiments, the administering of the albumin nanoparticle suspension to the subject or patient can be at a dose up to 320 mg per kilogram weight of the subject or patient.

While embodiments of the protein nanospheres and method to treat dysfunction from chemotherapy and immunosuppressive therapy have been described in detail, it should be apparent that modifications and variations thereto are possible, all of which fall within the true spirit and scope of the present technology. With respect to the above description then, it is to be realized that the optimum dimensional relationships for the parts of the present technology, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present technology. For example, any suitable sturdy material may be used instead of the above-described.

Therefore, the foregoing is considered as illustrative only of the principles of the present technology. Further, since numerous modifications and changes will readily occur to those skilled in the art, it is not desired to limit the present technology to the exact construction and operation shown and described, and accordingly, all suitable modifications and equivalents may be resorted to, falling within the scope of the present technology.

What is claimed as being new and desired to be protected by Letters Patent of the United States is as follows:

1. A method of using an albumin nanoparticle suspension containing submicron albumin spheres to mitigate damage to tissue caused by administration of a multi-valent agent to a subject in need thereof, the method comprising the step of administering intravenously a therapeutically effective amount of the albumin nanoparticle suspension containing the submicron albumin spheres to the subject, the albumin spheres being configured to augment a function or effectiveness of stem cells or precursor cells in vivo to mitigate damage to the tissue caused by treatment to the subject by the multi-valent agent.

2. The method according to claim 1, wherein the multi-valent agent has any one of or any combination of a chemotherapeutic effect and an immunosuppressive effect to the subject.

3. The method according to claim 1, wherein the albumin spheres are configured to have an effect on an oxidative reactivity of the stem cells or precursor cells.

4. The method according to claim 1, wherein the albumin spheres are configured to have an effect on the stem cells or precursor cells as an inducer of anti-oxidants or on an anti-oxidative pathway inside the stem cells or precursor cells.

5. The method according to claim 1, wherein the albumin spheres are configured to maintain a mass of a thymus of the subject and cells in the thymus.

6. The method according to claim 1, wherein the administering of the albumin nanoparticle suspension is prior to an onset of treatment of the multi-valent agent to the subject.

7. The method according to claim 1, wherein the administering of the albumin nanoparticle suspension is after an onset of treatment of the multi-valent agent to the subject.

8. The method according to claim 1, wherein the albumin spheres of the albumin nanoparticle suspension are bound with fibrinogen molecules to produce Fibrinogen Albumin Spheres (FAS).

9. The method according to claim 8, wherein the fibrinogen albumin spheres are High-Fibrinogen Spheres (HFS) prepared by coating blank albumin spheres with a solution containing human fibrinogen that is greater than 1.4 mg fibrinogen per mL of the solution.

10. The method according to claim 9, wherein the solution contains a concentration of sodium tetradecyl sulphate greater than 5 mg per mL of the solution, and wherein the sodium tetradecyl sulphate is configured to keep fibrinogen molecules in a soluble state without precipitation at room temperature.

11. The method according to claim 1, wherein the administering of the albumin nanoparticle suspension to the subject is at a dose greater than 160 mg per kilogram weight of the subject.

12. The method according to claim 1, wherein the administering of the albumin nanoparticle suspension to the subject is at a dose up to 320 mg per kilogram weight of the subject.

13. A method of treating one or more side-effects from administration of a multi-valent agent to a patient in need thereof with albumin nanoparticles, the method comprising the steps of:
a) providing a suspension including fibrinogen-coated albumin nanospheres prepared by coating blank albumin spheres with a solution containing human fibrinogen that is greater than 1.4 mg fibrinogen per mL of the solution; and
b) administering intravenously the suspension to the patient at a concentration of the fibrinogen-coated albumin nanospheres sufficient to augment a function or effectiveness of stem cells or precursor cells in vivo to at least mitigate damage caused by administration of the multi-valent agent to the patient.

14. The method according to claim 13, wherein the multi-valent agent has any one of or any combination of a chemotherapeutic effect and an immunosuppressive effect to the patient.

15. The method according to claim 13, wherein the albumin spheres are configured to have an effect selected from the group consisting of on an oxidative reactivity of the stem cells or precursor cells, on the stem cells or precursor cells as an inducer of anti-oxidants, and on an anti-oxidative pathway inside the stem cells or precursor cells.

16. The method according to claim 13, wherein the albumin spheres are configured to maintain a mass of a thymus of the patient and cells in the thymus.

17. The method according to claim 13, wherein the administering of the suspension is prior to an onset of treatment of the multi-valent agent to the patient.

18. The method according to claim 13, wherein the administering of the suspension is after an onset of treatment of the multi-valent agent to the patient.

19. The method according to claim 13, wherein the solution contains a concentration of sodium tetradecyl sulphate greater than 5 mg per mL of the solution, and wherein the sodium tetradecyl sulphate is configured to keep fibrinogen molecules in a soluble state without precipitation at room temperature.

20. The method according to claim 13, wherein the administering of the suspension to the patient is at a dose selected from the group consisting of greater than 160 mg per kilogram weight of the patient, and up to 320 mg per kilogram weight of the patient.
